(19) United States
(12) Patent Application Publication
(10) Pub. No.: US 2008/0152596 A1
(43) Pub. Date: Jun. 26, 2008

(54) Polypropylene glycol foamable vehicle and pharmaceutical compositions thereof

(75) Inventors: Doron Friedman, Karmei Yosef (IL); Dov Tamarkin, Maccabim (IL); Naomi Feiman, Raanana (IL); David Schuz, Moshav Gimzu (IL); Tal Berman, Rishon LeZiyyon (IL)

Correspondence Address: WILMERHALE/BOSTON, 60 STATE STREET, BOSTON, MA, 02109, US

(73) Assignee: Foamix Ltd., Ness Ziona (IL)

(21) Appl. No.: 11/894,767

(22) Filed: Aug. 20, 2007

Related U.S. Application Data
(63) Continuation of application No. 11/811,140, filed on Jun. 7, 2007.
Application No. 11/811,140 is a continuation-in-part of application No. 11/481,596, filed on Jul. 6, 2006.
Application No. 11/481,596 is a continuation-in-part of application No. 11/488,989, filed on Jul. 19, 2006.
Application No. 11/488,989 is a continuation-in-part of application No. 11/717,897, filed on Mar. 13, 2007.
(60) Provisional application No. 60/811,627, filed on Jun. 7, 2006.
(60) Provisional application No. 60/700,702, filed on Jul. 19, 2005.

(51) Int. Cl. A61K 9/00

(52) U.S. Cl. 424/43

(57) ABSTRACT

The present invention teaches a foamable pharmaceutical carrier comprising polypropylene glycol (PPG) alkyl ether, a surface-active agent water and a liquefied hydrocarbon gas propellant; and pharmaceutical compositions thereof.

The present invention further teaches a foamable pharmaceutical carrier comprising polypropylene glycol (PPG) alkyl ether, a surface-active agent, and a liquefied hydrocarbon gas propellant; and pharmaceutical compositions thereof.

### CROSS REFERENCE TO RELATED APPLICATIONS

This application claims the benefit of priority under 35 U.S.C. §120 to co-pending application Ser. No. 11/811,140, filed Jun. 7, 2007, which claims the benefit of priority under 35 U.S.C. §119(e) of U.S. Provisional Patent Application No. 60/811,627, filed on Jun. 7, 2006, entitled Polypropylene Glycol Foamable Vehicle and Pharmaceutical Compositions Thereof, which is incorporated herein by reference in its entirety.

This application is a continuation-in-part application of co-pending U.S. patent application Ser. No. 11/481,596, filed on Jul. 6, 2006, entitled “Non-Flammable Insecticide Compositon and Uses Thereof,” and is related to U.S. patent application Ser. No. 11/448,490, filed Jun. 7, 2006, entitled “Antibiotic Kit and Composition and Uses Thereof,” both of which are incorporated herein by reference in their entirety.

This application is a continuation-in-part application of co-pending U.S. patent application Ser. No. 11/488,989, filed on Jul. 19, 2006, which claims the benefit under 35 U.S.C. §119(e) of U.S. Provisional Patent Application No. 60/700,702, filed on Jul. 19, 2005, both entitled “Foamable Composition Combining a Polar Solvent and a Hydrophobic Carrier,” both of which are incorporated herein by reference in their entirety.

This application is a continuation-in-part application of co-pending U.S. patent application Ser. No. 11/717,897, filed on Mar. 13, 2007, entitled “Foamable Compositions, Kits and Methods for Hyperhidrosis,” which is incorporated herein by reference in its entirety.

### BACKGROUND OF THE INVENTION

This invention relates to foamable pharmaceutical and cosmetic compositions and foams, in particular poly(propylene) glycol (“PPG”) alkyl ether comprising foamable pharmaceutical and cosmetic compositions and foams.

External topical administration is an important route for the administration of drugs in disease treatment. Many groups of drugs, including, for example, antibiotic, anti-fungal, anti-inflammatory, anesthetic, analgesic, anti-allergic, corticosteroid, retinoid and anti-proliferative medications are preferably administered in hydrophobic media, namely ointment. However, ointments often form an impermeable barrier, so that metabolic products and excreta from the wounds to which they are applied are not easily removed or drained away. Furthermore, it is difficult for the active drug dissolved in the carrier to pass through the white petrolatum barrier layer into the wound tissue, so the efficacy of the drug is reduced. In addition, ointments and creams often do not create an environment for promoting respiration of the wound tissue and it is not favorable to the normal respiration of the skin. An additional disadvantage of petroleum jelly-based products relates to the greasy feeling left following their topical application onto the skin, mucosal membranes and wounds.

Foams are considered a more convenient vehicle for topical delivery of active agents. There are several types of topical foams, including aqueous foams, such as commonly available shaving foams; hydroalcoholic foams, such as described in U.S. Pat. No. 6,126,920; emulsion-based foams, comprising oil and water components, such as described in U.S. Pat. No. 6,730,288 and WO 2004/037225; and oleaginous foams, which consist of high oil content, such as described in U.S. Patent Application No. US 2005/0031547. In skin therapy, oil containing foams are preferred, since oil contributes to skin protection and moisturization, which improve the therapeutic effect of the formulation. Typically foams are made using liquefied hydrocarbon gas propellant, such a spropane, butane and isobutane.

As detailed hereinbelow, cosmetic formulations containing PPG ethers, such as PPG-15 stearyl ether, have been generally known. However, these formulations have only been known as creams, lotions or liquid formulations and have further been limited to a PPG content of up to 25%, more often up to 15%, and in most cases even much lower amounts of PPG (1-5%) reflecting the position that they are only listed in the FDA inactive ingredient list for topical ointments up to a concentration of 15%, and in the cosmetic field is allowed up to 25%, Yet further, albeit the many benefits of pharmaceutical and cosmetic foam formulations, until now no PPG-containing formulations have been developed in a foam form. In particular, foam formulations containing PPG-15 stearyl ether have not been expressly taught.

Thus for example, U.S. Pat. No. 6,001,341 discloses deodorant and/or antiperspirant cosmetic compositions comprising an alkyl ester or a mixture of alkyl esters wherein the carrying agents can be ethers of mono- and poly-hydroxylic alcohols or their mixtures such as, for example, dimethylisosorbide, di-isopropylether; polypropyleneglycol (PPG-10 cetyl ether, PPG-14 butyl ether, PPG-27 glyceryl ether). However, neither PPG-15 stearyl ether and/or foams thereof are exempified therein.

U.S. Pat. No. 5,614,178 discloses a water-based topical pharmaceutical composition having enhanced penetration through the skin, comprising from about 0.1% to about 25% of an alkoxylated ether, such as PPG-14 butyl ether, PPG-15 stearyl ether, and mixtures thereof, in addition to a safe and effective amount of a pharmaceutical active agent, from about 0.1% to about 10.0% of a high molecular weight crosslinked cationic polymer, and from about 0.05% to about 5% of a high HLB non-ionic surfactant. U.S. Pat. No. 5,614,178 does not teach or suggest preparing foams comprising PPG. Furthermore, U.S. Pat. No. 5,614,178 does not teach or suggest preparing compositions containing high amounts of PPG

WO 98/52536 discloses a skin care composition comprising a retinoid and a preservative, and further optionally comprising a carrier. PPG ethers are, inter alia, mentioned therein as possible ingredients in the carrier, but foams in general and PPG foams in particular, are neither taught nor suggested.

U.S. Pat. No. 4,083,974 discloses a topically applied pharmaceutical composition in an ointment form, which contain an effective amount of an anti-inflammatory steroid and 1-40% of polyoxypropylene 15 stearyl ether. Further are disclosed therein nonaqueous solutions and oinments for topical application, which comprise an effective amount of an anti-inflammatory steroid and a solubilizing effective amount of polyoxypropylene 15 stearyl ether. In one particular example, an ointment containing about 89% PPG-15 but no surfactant was prepared, but the application of this sample, as well as of any of the other ointments (having 15-40% PPG-15 stearate), on the skin—is not described. Furthermore, the preparation of foams of any of these compositions is neither taught nor suggested.

Whilst, U.S. Pat. No. 4,627,973 describes foam containing a unique combination of three moisturizers one of which is an alkoxylated methyl glucose derivative such as polypropylene glycol-20 methyl glucose, they are glucose derivatives and only small amounts (about 0.1 to 3% are claimed) and are used as part of the unique combination.

Other foamable compositions are described in: U.S. Publication No. 05-0232869, published on Oct. 20, 2005, entitled NONSTEROIDAL IMMUNOMODULATING KIT AND COMPOSITION AND USES THEREOF; U.S. Publication No. 05-0205086, published on Sep. 22, 2005, entitled RETINOID IMMUNOMODULATING KIT AND COMPOSITION AND USES THEREOF; U.S. Publication No. 06-0018937, published on Jan. 26, 2006, entitled STEROID KIT AND FOAMABLE COMPOSITION AND USES THEREOF; U.S. Publication No. 05-0271596, published on Dec. 8, 2005, entitled VASOACTIVE KIT AND COMPOSITION AND USES THEREOF; U.S. Publication No. 06-0269485, published on Nov. 30, 2006, entitled ANTIBIOTIC KIT AND COMPOSITION AND USES THEREOF; U.S. Publication No. 07-0020304, published on Jan. 25, 2007, entitled NON-FLAMMABLE INSECTICIDE COMPOSITION AND USES THEREOF; U.S. Publication No. 06-0193789, published on Aug. 31, 2006, entitled FILM FORMING FOAMABLE COMPOSITION; U.S. patent application Ser. No. 11/732,547, filed on Apr. 4, 2007, entitled ANTI-INFECTION AUGMENTATION OF FOAMABLE COMPOSITIONS AND KIT AND USES THEREOF; U.S. Provisional Patent Application No. 60/789,186, filed on Apr. 4, 2006, KERATOLYTIC ANTIFUNGAL FOAM; U.S. Provisional Patent Application No. 0/815948, filed on Jun. 23, 2006, entitled FOAMABLE COMPOSITIONS COMPRISING A CALCIUM CHANNEL BLOCKER, A CHOLINERGIC AGENT AND A NITRIC OXIDE DONOR; U.S. Provisional Patent Application No. 60/818,634, filed on Jul. 5, 2006, entitled DICARBOXYLIC ACID FOAMABLE VEHICLE AND PHARMACEUTICAL COMPOSITIONS THEREOF; U.S. Provisional Patent Application No. 60/843,140, filed on Sep. 8, 2006, entitled FOAMABLE VEHICLE AND VITAMIN PHARMACEUTICAL COMPOSITIONS THEREOF, all of which are incorporated herein by reference in their entirety.

There remains an unmet need for improved, easy to use, stable PPG-containing foamable formulations and foams, especially for treatment of dermal and mucosal tissues.

### SUMMARY OF INVENTION

The present invention relates to PPG alkyl ether comprising foamable compositions and foams.

According to one or more embodiments of the present invention, the foamable carrier, includes:
a polypropylene glycol alkyl ether;
a surface-active agent;
a solvent; and
a liquefied or compressed gas propellant at a concentration of about 3% to about 25% by weight of the total composition.

According to one or more embodiments of the present invention, the foamable carrier, includes:
a polypropylene glycol alkyl ether of about 3% to about 90% by weight of the total composition;
a surface-active agent;
a solvent; and
a liquefied or compressed gas propellant at a concentration of about 3% to about 25% by weight of the total composition.

According to one or more embodiments of the present invention, the foamable therapeutic composition includes:
a therapeutically effective amount of an active agent;
a polypropylene glycol alkyl ether of about 3% to about 90% by weight of the total composition;
a surface-active agent;
a solvent; and
a liquefied or compressed gas propellant at a concentration of about 3% to about 25% by weight of the total composition.

According to one or more embodiments of the present invention, the method of treating a disorder of a mammalian subject, includes:
administering a foamable therapeutic composition to a target site, the composition comprising:
a therapeutically effective concentration of an active agent;
a polypropylene glycol (PPG) alkyl ether of about 3% to about 90% by weight of the total composition;
a surface-active agent;
a solvent; and
a liquefied or compressed gas propellant at a concentration of about 3% to about 25% by weight of the total composition.

According to one or more embodiments of the present invention, the foamable pharmaceutical carrier includes:
at least 15% polypropylene glycol alkyl ether and liquefied or compressed gas propellant at a concentration of about 3% to about 25% by weight of the total composition.

According to one or more embodiments of the present invention, the foamable therapeutic composition includes:
a therapeutically effective amount of an active agent;
at least 15% polypropylene glycol alkyl ether and
a liquefied or compressed gas propellant at a concentration of about 3% to about 25% by weight of the total composition.

According to one or more embodiments of the present invention, the method of treating a disorder of a mammalian subject includes:
administering a foamable therapeutic composition to a target site, the composition comprising:
   a therapeutically effective concentration of an active agent;
   at least 15% polypropylene glycol alkyl ether and
   a liquefied or compressed gas propellant at a concentration of about 3% to about 25% by weight of the total composition.

### FIGURES

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a microscope picture of an exemplary formulation B, according to a preferred embodiment of the present invention, which contains about 60% PPG and about 33% water; and

FIG. 2 is a microscope picture of an exemplary formulation A, according to a preferred embodiment of the present invention, which contains about 80% PPG and no water.

### DESCRIPTION OF THE INVENTION

The present invention relates to a composition for use as foamable vehicle composition.

According to one or more embodiments of the present invention, the foamable carrier, includes:
a) a polypropylene glycol alkyl ether;
b) a surface-active agent;
c) a solvent; and
d) a liquefied hydrocarbon gas propellant at a concentration of about 3% to about 25% by weight of the total composition.

According to one or more embodiments the present invention includes the embodiments described above in the Summary of the invention as more particularly exemplified below,

All % values are provided on a weight (w/w) basis.

In one or more embodiments the foamable vehicle further includes a foam adjuvant. More particularly the foam ajuvant is preferably selected from the group consisting of a fatty alcohol; a fatty acid; and a fatty alcohol.

Optionally, the foamable vehicle further includes at least one organic carrier selected from the group consisting of a hydrophobic organic carrier, an organic polar solvent, an emollient and mixtures thereof, at a concentration of about 2% to about 50% by weight. The hydrophobic solvent and/or the emollient can be selected from the group consisting of mineral oil, alkyl esters of fatty acids such as isopropyl palmitate, isopropyl isostearate, diisopropyl adipate, diisopropyl dimerate, octyl palmitate, cetyl lactate, cetyl ricinoleate, tocopheryl acetate, acetylated lanolin alcohol, cetyl acetate, phenyl trimethicone, glyceryl oleate, tocopheryl linoleate, wheat germ glycerides, arachidyl propionate, myristyl lactate, decyl oleate, ricinoleate, isopropyl lanolate, pentaerythrityl tetrastearate, neopentylglycol dicaprylate/dicaprate, isononyl isononanoate, isotridecyl isononanoate, myristyl myristate, triisocetyl citrate, octyl dodecanol, maleated soybean oil, unsaturated or polyunsaturated oils, such as olive oil, corn oil, soybean oil, canola oil, cottonseed oil, coconut oil, sesame oil, sunflower oil, borage seed oil, syzigium aromaticum oil, hempseed oil, herring oil, cod-liver oil, salmon oil, flaxseed oil, wheat germ oil, evening primrose oils; essential oils; and silicone oils, such as dimethicone, cyclomethicone, polyalkyl siloxane, polyaryl siloxane, polyalkylaryl siloxane, a polyether siloxane copolymer and a poly(dimethylsiloxane)-(diphenyl-siloxane) copolymer. A “polar solvent” is an organic solvent, typically soluble in both water and oil. Examples of polar solvents include polyols, such as glycerol (glycerin), propylene glycol, hexylene glycol, diethylene glycol, propylene glycol n-alkanols, terpenes, di-terpenes, tri-terpenes, terpen-ols, limonene, terpene-ol, 1-menthol, dioxolane, ethylene glycol, other glycols, sulfoxides, such as dimethylsulfoxide (DMSO), dimethylformanide, methyl dodecyl sulfoxide, dimethylacetamide, azone (1-dodecylazacycloheptan-2-one), 2-(n-nonyl)-1,3-dioxolane, alkanols, such as dialkylamino acetates, and admixtures thereof.

### Polypropylene Glycol (PPG) Alkyl Ethers

In the context of the present invention, a polypropylene glycol alkyl ether (PPG alkyl ether) is a liquid, water-insoluble propoxylated fatty alcohol, having the molecular formula of RO(CH₂CHOCH₃)ₙ; wherein “R” is a straight-chained or branched C₄ to C₂₂ alkyl group; and “n” is in the range between 4 and about 50.

(PPG alkyl ethers), are organic liquids that function as skin-conditioning agent in pharmaceutical and cosmetic formulations. They possess exceptional emollient effect, side by side with enhanced solvency properties, which facilitates solubilization of active agents in a composition comprising a PPG alkyl ether. PPG alkyl ethers offer the following advantages when used as a component in the foamable composition of the present invention:
Due to the polypropylene glycol moiety, PPG alkyl ethers possess certain surface active properties and they assist in the coupling of polar and non-polar oils in an emulsion formulation.
PPG alkyl ethers are non-occlusive; offering a long-lasting and velvety feel.
They are chemically stable at extreme pH conditions;
Excellent solvency properties, particularly with difficult to formulate active agents
When combined with certain surfactants, such as Brij 72 and Brij 721, PPG alkyl ethers form oleosomes and/or liquid crystal structures, which provide long lasting moisturization, excellent spreading as well as prolonged hydration properties

Exemplary PPG alkyl ethers include PPG-2 butyl ether, PPG-4 butyl ether, PPG-5 butyl ether, PPG-9 butyl ether, PPG-12 butyl ether, PPG-14 butyl ether, PPG-15 butyl ether, PPG-16 butyl ether, PPG-17 butyl ether, PPG-18 butyl ether, PPG-20 butyl ether, PPG-22 butyl ether, PPG-24 butyl ether, PPG-26 butyl ether, PPG-30 butyl ether, PPG-33 butyl ether, PPG-40 butyl ether, PPG-52 butyl ether, PPG-53 butyl ether, PPG-10 cetyl ether, PPG-28 cetyl ether, PPG-30 cetyl ether, PPG-50 cetyl ether, PPG-30 isocetyl ether, PPG-4 lauryl ether, PPG-7 lauryl ether, PPG-2 methyl ether, PPG-3 methyl ether, PPG-3 myristyl ether, PPG-4 myristyl ether, PPG-10 oleyl ether, PPG-20 oleyl ether, PPG-23 oleyl ether, PPG-30 oleyl ether, PPG-37 oleyl ether, PPG-50 oleyl ether, PPG-11 stearyl ether. Preferred PPG alky ethers according to the present invention include PPG-15 stearyl ether (also known as Earlamol E®, Unichema), PPG-2 butyl ether, PPG-9-13 butyl ether and PPG-40 butyl ether. PPG alkyl ethers can be incorporated in the foamable composition of the present invention in a concentration between about 1% and about 90%, more preferably above 15%, above 20%, above 30% . . . and up to 60% PPG.

It has been discovered that when the solvent is aqueous, the PPG content cannot be increased beyond about 60% without losing the foam structure.
The ability to achieve concentrations as high as

The sensory properties of foams containing PPG alkyl ethers are favorable, as revealed by consumer panel tests (see for example, Table XX below).

Surprisingly, it has been discovered that PPG alkyl ethers also reduce the degree of inflammability of a foam, as demonstrated in a standard inflammability test according to European Standard prEN 14851, titled “Aerosol containers-Aerosol foam flammability test” was performed on foam compositions PPG 1 and PPG 5. According to this standard, a product is considered inflammable if a stable flame appears following ignition, which is at least 4 cm high and which is maintained for at least 2 seconds. Thus, in an embodiment of the present invention the foamable composition, which contains a PPG alkyl ether is non-flammable, when tested according to European Standard prEN 14851. In additional embodiments, the concentration of the PPG alkyl ether is sufficient to reduce the degree of inflammability, when compared with the same composition where the oil component comprises an another oil, such as mineral oil or an ester of a fatty acid.

### PPG Stearyl Ethers

PPG stearyl ethers function as skin-conditioning and penetration agents in cosmetic formulations.

Polypropylene glycol stearyl ether 15, also known as polyoxypropylene 15 stearyl ether or as “PPG-15”, and having a CAS Registry No. of [25231-21-4], is a stearyl ether having about 15 propylene oxide units incorporated in its structure. PPG-15 stearyl ether is a clear liquid, soluble in mineral oil, isopropyl ethers, cottonseed oil, ethanol, isopropanol and hexadecyl alcohol, to name a few, and is particularly useful as a solvent of difficult to formulate ingredients, such as sunscreens, aluminum chiorhydrate salts and skin toners. It is insoluble in water, propylene glycol and glycerin. PPG-15 stearyl ether is an inert and highly stable compound.

PPG stearyl ether has been known to form liquid crystal structures known as “oleosomes”, which are oil-in-water emulsions having multiple layers of water, emollient and emulsifier. Such structures may offer several benefits in the preparation of cosmetical or pharmaceutical formulations in that they improve the dissolution of poorly water-soluble drug, the ability to control the release of drugs and/or the ability to protect and facilitate the transport of “fragile” molecules.

PPG stearyl ether also functions as a coupling agent, allowing, for example, the compatibility of polar and non polar oils with ethanol and perfumes in after shave lotions. It is chemically stable at extreme pH levels and at the same time saturated, providing excellent shelf life stability. Because they are such powerful emulsion stabilizers, oleosomes are effective to formulate ingredients such as salts (as in the case of the antiperspirant), extreme pH (found in formulations using alpha and beta hydroxy acids and depilatory formulations) or formulations requiring a high level of alcohol (such as refreshing body milk or aftershaves).

Furthermore, the extra layers of water and oil in an oleosome offers noticeable benefits in the areas of skin feel and moisturization. Oleosomes have a luxurious feel when introduced to the skin and excellent subsequent rubout characteristics. The bound water in the oleosome offers long lasting moisturization potential as well.

### Polymeric Agent

The composition of the present invention contains a polymeric agent selected from the group consisting of a bioadhesive agent, a gelling agent, a film forming agent and a phase change agent. A polymeric agent enhances the creation of foam having fine bubble structure, which does not readily collapse upon release from the pressurized aerosol can. The polymeric agent serves to stabilize the foam composition and to control drug residence in the target organ.

Exemplary polymeric agents include, in a non-limiting manner, naturally-occurring polymeric materials, such as locust bean gum, sodium alginate, sodium caseinate, egg albumin, gelatin agar, carrageenin gum, sodium alginate, xanthan gum, quince seed extract, tragacanth gum, guar gum, cationic guars, hydroxypropyl guar gum, starch, amine-bearing polymers such as chitosan; acidic polymers obtainable from natural sources, such as alginic acid and hyaluronic acid; chemically modified starches and the like, carboxyvinyl polymers, polyvinylpyrrolidone, polyvinyl alcohol, polyacrylic acid polymers, polymethacrylic acid polymers, polyvinyl acetate polymers, polyvinyl chloride polymers, polyvinylidene chloride polymers and the like.

Additional exemplary polymeric agents include semi-synthetic polymeric materials such as cellulose ethers, such as methylcellulose, hydroxypropyl cellulose, hydroxypropyl methylcellulose, hydroxyethyl cellulose, hydroxy propylmethyl cellulose, methylhydroxyethylcellulose, methylhydroxypropylcellulose, hydroxyethylcarboxymethylcellulose, carboxymethyl cellulose, carboxymethylcellulose carboxymethylhydroxyethylcellulose, and cationic celluloses, carbomer (homopolymer of acrylic acid is crosslinked with an allyl ether pentaerythritol, an allyl ether of sucrose, or an allyl ether of propylene, such as Carbopol® 934, Carbopol® 940, Carbopo® 941, Carbopol® 980 and Carbopol® 981. Polyethylene glycol, having molecular weight of 1000 or more (e.g., PEG 1,000, PEG 4,000, PEG 6,000 and PEG 10,000) also have gelling capacity and while they are considered herein as “secondary polar solvents”, as detailed herein, they are also considered polymeric agents.

Mixtures of the above polymeric agents are contemplated.

The concentration of the polymeric agent should be selected so that the composition, after filling into aerosol canisters, is flowable, and can be shaken in the canister. In one or more embodiments, the concentration of the polymeric agent is selected such that the viscosity of the composition, prior to filling of the composition into aerosol canisters, is less than 12,000 CPs, and more preferably, less than 10,000 CPs.

### Surface Active Agent

The composition of the present invention further contains a surface-active agent. Surface-active agents (also termed “surfactants”) include any agent linking oil and water in the composition, in the form of emulsion. A surfactant's hydrophilic/lipophilic balance (HLB) describes the emulsifier's affinity toward water or oil. HLB is defined for non-ionic surfactants. The HLB scale ranges from 1 (totally lipophilic) to 20 (totally hydrophilic), with 10 representing an equal balance of both characteristics. Lipophilic emulsifiers form water-in-oil (w/o) emulsions; hydrophilic surfactants form oil-in-water (o/w) emulsions. The HLB of a blend of two emulsifiers equals the weight fraction of emulsifier A times its HLB value plus the weight fraction of emulsifier B times its HLB value (weighted average). In many cases a single surfactant may suffice. In other cases a combination of two or more surfactants is desired. Reference to a surfactant in the specification can also apply to a combination of surfactants or a surfactant system. As will be appreciated by a person skilled in the art which surfactant or surfactant system is more appropriate is related to the vehicle and intended purpose. In general terms a combination of surfactants is usually preferable where the vehicle is an emulsion. In an emulsion environment a combination of surfactants can be significant in producing breakable forms of good quality. It has been further discovered that the generally thought considerations for HLB values for selecting a surfactant or sufactant combination are not always binding for emulsions and that good quality foams can be produced with a surfactant or surfactant combination both where the HLB values are in or towards the lipophilic side of the scale and where the HLB values are in or towards the hydrophilic side of the scale. Surfactants also play a role in foam formation where the foamable formulation is a single phase composition.

According to one or more embodiments the composition contains a single surface active agent having an HLB value between about 2 and 9, or more than one surface active agent and the weighted average of their HLB values is between about 2 and about 9. Lower HLB values may in certain embodiments be more applicable to water in oil emulsions.

According to one or more embodiments the composition contains a single surface active agent having an HLB value between about 7 and 14, or more than one surface active agent and the weighted average of their HLB values is between about 7 and about 14. Mid range HLB values may in certain embodiments be more suitable for oil in water emulsions.

According to one or more other embodiments the composition contains a single surface active agent having an HLB value between about 9 and about 19, or more than one surface active agent and the weighted average of their HLB values is between about 9 and about 19. In a waterless or substantially waterless environment a wide range of HLB values may be suitable.

Preferably, the composition of the present invention contains a non-ionic surfactant. Nonlimiting examples of possible non-ionic surfactants include a polysorbate, polyoxyethylene (20) sorbitan monostearate, polyoxyethylene (20) sorbitan monooleate, a polyoxyethylene fatty acid ester, Myrj 45, Myrj 49, Myrj 52 and Myrj 59; a polyoxyethylene alkyl ether, polyoxyethylene cetyl ether, polyoxyethylene palmityl ether, polyethylene oxide hexadecyl ether, polyethylene glycol cetyl ether, steareths such as steareth 2, brij 21, brij 721, brij 38, brij 52, brij 56 and brij W1, a sucrose ester, a partial ester of sorbitol and its anhydrides, sorbitan monolaurate, sorbitan monolaurate, a monoglyceride, a diglyceride, isoceteth-20 and mono-, di- and tri-esters of sucrose with fatty acids. In certain embodiments, suitable sucrose esters include those having high monoester content, which have higher HLB values.

Non-limiting examples of non-ionic surfactants that have HLB of about 7 to about 12 include steareth 2 (HLB˜4.9); glyceryl monostearate/PEG 100 stearate (Av HLB˜11.2); stearate Laureth 4 (HLB˜9.7) and cetomacrogol ether (e.g., polyethylene glycol 1000 monocetyl ether).

Non-limiting examples of preferred surfactants, which have a HLB of 4-19 are set out in the Table below:

| Surfactant | HLB |
|---|---|
| steareth 2 | ~4.9 |
| glyceryl monostearate/PEG 100 stearate | Av ~11.2 |
| Glyceryl Stearate | ~4 |
| Steareth 21 | ~15.5 |
| peg 40 stearate | ~16.9 |
| polysorbate 80 | ~15 |
| sorbitan stearate | ~4.7 |
| laureth 4 | ~9.7 |
| Sorbitan monooleate (span 80) | ~4.3 |
| ceteareth 20 | ~15.7 |
| steareth 20 | ~15.3 |
| ceteth 20 | ~15.7 |
| Macrogol Cetostearyl Ether | ~15.7 |
| ceteth 2 (Lipocol C-2) | ~5.3 |
| PEG-30 Dipolyhydroxystearate | ~5.5 |
| sucrose distearate (Sisterna SP30) | ~6 |
| polyoxyethylene (100) stearate | ~18.8 |

More exemplary stabilizing surfactants which may be suitable for use in the present invention are found below.

### PEG-Fatty Acid Monoester Surfactants

| Chemical name | Product example name | HLB |
|---|---|---|
| PEG-30 stearate | Myrj 51 | >10 |
| PEG-40 laurate | Crodet L40 (Croda) | 17.9 |
| PEG-40 oleate | Crodet O40 (Croda) | 17.4 |
| PEG-45 stearate | Nikkol MYS-45 (Nikko) | 18 |
| PEG-50 stearate | Myrj 53 | >10 |
| PEG-100 stearate | Myrj 59, Arlacel 165 (ICI) | 19 |

### PEG-Fatty Acid Diester Surfactants:

| Chemical name | Product example name | HLB |
|---|---|---|
| PEG-4 dilaurate | Mapeg .RTM. 200 DL (PPG), | 7 |
| | Kessco .RTM.PEG 200 DL | |
| | (Stepan), LIPOPEG 2-DL (Lipo | |
| | Chem.) | |
| PEG-4 | distearate Kessco .RTM. 200 | 5 |
| | DS (Stepan.sub) | |
| PEG-32 dioleate | Kessco .RTM. PEG 1540 DO | 15 |
| | (Stepan) | |
| PEG-400 dioleate | Cithrol 4DO series (Croda) | >10 |
| PEG-400 disterate | Cithrol 4DS series (Croda) | >10 |
| PEG-20 glyceryl oleate | Tagat .RTM. O (Goldschmidt) | >10 |

### Transesterification Products of Oils and Alcohols

| Chemical name | Product example name | HLB |
|---|---|---|
| PEG-30 castor oil | Emalex C-30 (Nihon Emulsion) | 11 |
| PEG-40 hydrogenated | Cremophor RH 40 (BASF), | 13 |
| castor oil | Croduret (Croda), Emulgin | |
| | HRE 40 (Henkel) | |

Polyglycerized Fatty Acids, such as:

| Chemical name | Product example name | HLB |
|---|---|---|
| Polyglyceryl-6 dioleate | Caprol .RTM. 6G20 (ABITEC); | 8.5 |
| | PGO-62 (Calgene), PLUROL | |
| | OLEIQUE CC 497 | |
| | (Gattefosse)Hodag | |

### PEG-Sorbitan Fatty Acid Esters

| Chemical name | Product example name | HLB |
|---|---|---|
| PEG-20 sorbitan | Tween-20 (Atlas/ICI), Crillet 1 | 17 |
| monolaurate | (Croda), DACOL MLS 20 | |
| | (Condea) | |
| PEG-20 sorbitan | Tween 40 (Atlas/ICI), Crillet 2 | 16 |
| Monopalmitate | (Croda) | |
| PEG-20 sorbitan | Tween-60 (Atlas/ICI), Crillet 3 | 15 |
| monostearate | (Croda) | |
| PEG-20 sorbitan | Tween-80 (Atlas/ICI), Crillet 4 | 15 |
| monooleate | (Croda) | |

### Polyethylene Glycol Alkyl Ethers

| Chemical name | Product example name | HLB |
|---|---|---|
| PEG-2 oleyl ether | oleth-2 Brij 92/93 (Atlas/ICI) | 4.9 |
| PEG-3 oleyl ether | oleth-3 Volpo 3 (Croda) | <10 |
| PEG-5 oleyl ether | oleth-5 Volpo 5 (Croda) | <10 |
| PEG-10 oleyl ether | oleth-10 Volpo 10 (Croda), Brij | 12 |
| | 96/97 (Atlas/ICI) | |
| PEG-20 oleyl ether | oleth-20 Volpo 20 (Croda), Brij | 15 |
| | 98/99 (Atlas/ICI) | |
| PEG-4 lauryl ether | laureth-4Brij 30 (Atlas/ICI) | 9.7 |
| PEG-23 lauryl ether | laureth-23Brij 35 (Atlas/ICI) | 17 |
| PEG-10 stearyl ether | Brij 76 (ICI) | 12 |
| PEG-2 cetyl ether | Brij 52 (ICI) | 5.3 |

### Sugar Ester Surfactants

| Chemical name | Product example name | HLB |
|---|---|---|
| Sucrose distearate | Sisterna SP50, Surfope 1811 | 11 |

### Sorbitan Fatty Acid Ester Surfactants

| Chemical name | Product example name | HLB |
|---|---|---|
| Sorbitan monolaurate | Span-20 (Atlas/ICI), Crill 1 | 8.6 |
| | (Croda), Arlacel 20 (ICI) | |
| Sorbitan monopalmitate | Span-40 (Atlas/ICI), Crill 2 | 6.7 |
| | (Croda), Nikkol SP-10 (Nikko) | |
| Sorbitan monooleate | Span-80 (Atlas/ICI), Crill 4 | 4.3 |
| | (Croda), Crill 50 (Croda) | |
| Sorbitan monostearate | Span-60 (Atlas/ICI), Crill 3 | 4.7 |
| | (Croda), Nikkol SS-10 (Nikko) | |

In one or more embodiments the surface active agent is a complex emulgator in which the combination of two or more surface active agents can be more effective than a single surfactant and provides a more stable emulsion or improved foam quality than a single surfactant. For example and by way of non-limiting explanation it has been found that by choosing say two surfactants, one hydrophobic and the other hydrophilic the combination can produce a more stable emulsion than a single surfactant. Preferably, the complex emulgator comprises a combination of surfactants wherein there is a difference of about 4 or more units between the HLB values of the two surfactants or there is a significant difference in the chemical nature or structure of the two or more surfactants.

Specific non limiting examples of surfactant systems are, combinations of polyoxyethylene alkyl ethers, such as Brij 59, Brij10; Brij 52/Brij 10; Steareth 2/Steareth 20; Steareth 2/Steareth 21 (Brij 72/Brij 721); combinations of polyoxyethylene stearates such as Myrj 52/Myrj 59; combinations of sucrose esters, such as Surphope 1816/Surphope 1807; combinations of sorbitan esters, such as Span 20/Span 80; Span 20/Span 60; combinations of sucrose esters and sorbitan esters, such as Surphope 1811 and Span 60; combinations of liquid polysorbate detergents and PEG compounds, such as Tween 80/PEG-40 stearate; methyl glucaso sequistearate; polymeric emulsifiers, such as Permulen (TRI or TR2); liquid crystal systems, such as Arlatone (2121), Stepan (Mild RM1), Nikomulese (41) and Montanov (68) and the like.

In certain embodiments the surfactant is preferably one or more of the following: a combination of steareth-2 and steareth-21 on their own or in combination with GMS; in certain other embodiments the surfactant is a combination of polysorbate 80 and PEG-40 stearate. In certain other embodiments the surfactant is a combination of glyceryl monostearate/PEG 100 stearate. In certain other embodiments the surfactant is a combination of two or more of stearate 21, PEG 40 stearate, and polysorbate 80. In certain other embodiments the surfactant is a combination of two or more of laureth 4, span 80, and polysorbate 80. In certain other embodiments the surfactant is a combination of two or more of GMS and ceteareth. In certain other embodiments the surfactant is a combination of two or more of steareth 21, ceteareth 20, ceteth 2 and laureth 4 In certain other embodiments the surfactant is a combination of ceteareth 20 and polysorbate 40 stearate. In certain other embodiments the surfactant is a combination of span 60 and GMS.

In one or more embodiments the stability of the composition can be improved when a combination of at least one non-ionic surfactant having HLB of less than 9 and at least one non-ionic surfactant having HLB of equal or more than 9 is employed. The ratio between the at least one non-ionic surfactant having HLB of less than 9 and the at least one non-ionic surfactant having HLB of equal or more than 9, is between 1:8 and 8:1, or at a ratio of 4:1 to 1:4. The resultant HLB of such a blend of at least two emulsifiers is preferably between about 9 and about 14.

Thus, in an exemplary embodiment, a combination of at least one non-ionic surfactant having HLB of less than 9 and at least one non-ionic surfactant having HLB of equal or more than 9 is employed, at a ratio of between 1:8 and 8:1, or at a ratio of 4:1 to 1:4, wherein the HLB of the combination of emulsifiers is preferably between about 5 and about 18.

In certain cases, the surface active agent is selected from the group of cationic, zwitterionic, amphoteric and ampholytic surfactants, such as sodium methyl cocoyl taurate, sodium methyl oleoyl taurate, sodium lauryl sulfate, triethanolamine lauryl sulfate and betaines.

Many amphiphilic molecules can show lyotropic liquid-crystalline phase sequences depending on the volume balances between the hydrophilic part and hydrophobic part. These structures are formed through the micro-phase segregation of two incompatible components on a nanometer scale. Soap is an everyday example of a lyotropic liquid crystal. Certain types of surfactants tend to form lyotropic liquid crystals in emulsions interface (oil-in-water) and exert a stabilizing effect. Non limiting examples of surfactants with postulated tendency to form interfacial liquid crystals are: phospholipids, alkyl glucosides, sucrose esters, sorbitan esters. In certain embodiments of the present invention surfactants which tend to form liquid crystals may improve the quality of foams produced from compositions of the present invention.

In one or more embodiments the surfactant is a surfactant or surfactant combination is capable of or which tends to form liquid crystals.

In one or more embodiments the at least one surface active agent is liquid.

In one or more embodiments the at least one surface active agent is solid, semi solid or waxy.

It should be noted that HLB values may not be so applicable to non ionic surfactants, for example, with liquid crystals or with silicones. Also HLB values may be of lesser significance in a waterless or substantially non-aqueous environment.

In one or more embodiments the surfactant can be, a surfactant system comprising of a surfactant and a co surfactant, a waxy emulsifier, a liquid crystal emulsifier, an emulsifier which is solid or semi solid at room temperature and pressure, or combinations of two or more agents in an appropriate proportion as will be appreciated a person skilled in the art. Where a solid or semi solid emulsifier combination is used it can also comprise a solid or semi solid emulsifier and a liquid emulsifier.

In one or more embodiments of the present invention, the surface-active agent includes at least one non-ionic surfactant. Ionic surfactants are known to be irritants. Therefore, non-ionic surfactants are preferred in applications including sensitive tissue such as found in most mucosal tissues, especially when they are infected or inflamed. We have surprisingly found that non-ionic surfactants alone can provide formulations and foams of good or excellent quality in the carriers and compositions of the present invention.

Thus, in a preferred embodiment, the surface active agent, the composition contains a non-ionic surfactant. In another preferred embodiment the composition includes a mixture of non-ionic surfactants as the sole surface active agent. Yet, in additional embodiments, the foamable composition includes a mixture of at least one non-ionic surfactant and at least one ionic surfactant in a ratio in the range of about 100:1 to 6:1. In one or more embodiments, the non-ionic to ionic surfactant ratio is greater than about 6:1, or greater than about 8:1; or greater than about 14:1, or greater than about 16:1, or greater than about 20:1. In further embodiments, surface active agent comprises a combination of a non-ionic surfactant and an ionic surfactant, at a ratio of between 1:1 and 20:1.

In one or more embodiments of the present invention, a combination of a non-ionic surfactant and an ionic surfactant (such as sodium lauryl sulphate and cocamidopropylbetaine) is employed, at a ratio of between 1:1 and 20:1, or at a ratio of 4:1 to 10:1; for example, about 1:1, about 4:1, about 8:1, about 12:1, about 16:1 and about 20:1 or at a ratio of 4:1 to 10:1, for example, about 4:1, about 6:1, about 8:1 and about 10:1.

In selecting a suitable surfactant or combination thereof it should be borne in mind that the upper amount of surfactant that may be used may be limited by the shakability of the composition. In general terms, as the amount of non liquid surfactant is increased the shakability of the formulation reduces until a limitation point is reached where the formulation becomes non shakable and unsuitable. Thus in an embodiment of the present invention any effective amount of surfactant may be used provided the formulation remains shakable. In other certain exceptional embodiments the upper limit may be determined by flowability such as in circumstances where the composition is marginally or apparently non shakable. Thus in an embodiment of the present invention any effective amount of surfactant may be used provided the formulation remains flowable.

In certain embodiments of the present invention the amount of surfactant or combination of surfactants is between about 0.05% to about 20%; between about 0.05% to about 15%. or between about 0.05% to about 10%. In a preferred embodiment the concentration of surface active agent is between about 0.2% and about 8%. In a more preferred embodiment the concentration of surface active agent is between about 1% and about 6%.

If the composition as formulated is a substantially non shakable composition it is nevertheless possible as an exception in the scope of the present invention for the formulation to be flowable to a sufficient degree to be able to flow through an actuator valve and be released and still expand to form a good quality foam. This surprising and unusual exception may be due one or more of a number of factors such as the high viscosity, the softness, the lack of crystals, the pseudoplastic or semi pseudo plastic nature of the composition and the dissolution of the propellant into the petrolatum.

In one or more embodiments of the present invention, the surface-active agent includes mono-, di- and tri-esters of sucrose with fatty acids (sucrose esters), prepared from sucrose and esters of fatty acids or by extraction from sucro-glycerides. Suitable sucrose esters include those having high monoester content, which have higher HLB values.

### Substantially Alcohol-Free

According to one or more embodiments, the foamable composition is substantially alcohol-free, i.e., free of short chain alcohols. Short chain alcohols, having up to 5 carbon atoms in their carbon chain skeleton and one hydroxyl group, such as ethanol, propanol, isopropanol, butaneol, iso-butaneol, t-butaneol and pentanol, are considered less desirable solvents or polar solvents due to their skin-irritating effect. Thus, the composition is substantially alcohol-free and includes less than about 5% final concentration of lower alcohols, preferably less than about 2%, more preferably less than about 1%.

In certain cases, the active agent degrades in the presence of water, and therefore, in such cases the present of water in the composition is not desirable. Thus, in certain preferred embodiments, the composition is substantially non-aqueous. The term “substantially non-aqueous” or “substantially waterless” is intended to indicate that the composition has a water content below about 5%, preferably below about 2%, such as below about 1.5%. In certain other preferred embodiments the composition is non aqueous or waterless.

By non aqueous or waterless is meant that the composition contains no or substantially no, free or unassociated or absorbed water. It will be understood by a person of the art that the waterless solvents and substances miscible with them of the present invention can be hydrophilic and can contain water in an associated or unfree or absorbed form and may absorb water from the atmosphere and the ability to do so is its hygroscopic water capacity. It is intended that essentially non-aqueous formulations are included within its scope such that the formulations may have present a small amount of water. In some embodiments the composition ingredients are pretreated to reduce, remove or eliminate any residual or associated or absorbed water.

### Shakability

‘Shakability’ means that the composition contains some or sufficient flow to allow the composition to be mixed or remixed on shaking. That is, it has fluid or semi fluid properties. In some very limited cases possibly aided by the presence of silicone it may exceptionally be possible to have a foamable composition which is flowable but not apparently shakable.

A breakable foam is one that is thermally stable, yet breaks under sheer force.

### Breakability

The breakable foam of the present invention is not “quick breaking”, i.e., it does not readily collapse upon exposure to body temperature environment. Sheer-force breakability of the foam is clearly advantageous over thermally induced breakability, since it allows comfortable application and well directed administration to the target area.

### Additional Components

In an embodiment of the present invention, a composition of the present invention includes one or more additional components. Such additional components include but are not limited to anti perspirants, anti-static agents, buffering agents, bulking agents, chelating agents, cleansers, colorants, conditioners, deodorants, diluents, dyes, emollients, fragrances, hair conditioners, humectants, pearlescent aids, perfuming agents, permeation enhancers, pH-adjusting agents, preservatives, protectants, skin penetration enhancers, softeners, solubilizers, sunscreens, sun blocking agents, sunless tanning agents, viscosity modifiers and vitamins. As is known to one skilled in the art, in some instances a specific additional component may have more than one activity, function or effect.

### Propellants

Suitable propellants include volatile hydrocarbons such as butane, propane, isobutane and fluorocarbon gases, or mixtures thereof.

The propellant makes up about 3-25 wt % of the foamable composition. The propellants are used to generate and administer the foamable composition as a foam. The total composition including propellant, foamable compositions and optional ingredients is referred to as the foamable composition.

Alcohol and organic solvents render foams inflammable. It has been surprisingly discovered that fluorohydrocarbon propellants, other than chloro-fluoro carbons (CMCs), which are non-ozone-depleting propellants, are particularly useful in the production of a non-flammable foamable composition. A test according to European Standard prEN 14851, titled “Aerosol containers-Aerosol foam flammability test” revealed that compositions containing an organic carrier that contains a hydrophobic organic carrier and/or a polar solvent, which are detected as inflammable when a hydrocarbon propellant is used, become non-flammable, while the propellant is an HFC propellant.

Such propellants include, but are not limited to, hydrofluorocarbon (HFC) propellants, which contain no chlorine atoms, and as such, fall completely outside concerns about stratospheric ozone destruction by chlorofluorocarbons or other chlorinated hydrocarbons. Exemplary non-flammable propellants according to this aspect of the invention include propellants made by DuPont under the registered trademark Dymel, such as 1,1,1,2 tetrafluorethane (Dymel 134), and 1,1,1,2,3,3,3 heptafluoropropane (Dymel 227). HFCs possess Ozone Depletion Potential of 0.00 and thus, they are allowed for use as propellant in aerosol products.

Notably, the stability of foamable emulsions including HFC as the propellant can be improved in comparison with the same composition made with a hydrocarbon propellant.

In one or more embodiments foamable compositions comprise a combination of a HFC and a hydrocarbon propellant such as n-butanee or mixtures of hydrocarbom propellants such as propane, ispbutane and butane.

Suitable propellants include volatile hydrocarbons such as butane, propane, isobutane and fluorocarbon gases, or mixtures thereof.

The propellant makes up about 5-25 wt % of the foamable composition. The propellants are used to generate and administer the foamable composition as a foam. The total composition including propellant, foamable compositions and optional ingredients is referred to as the foamable composition.

### Modulating Agent

The term modulating agent is used to describe an agent which can improve the stability of or stabilize a foamable carrier or composition and or an active agent by modulating the effect of a substance or residue present in the carrier or composition.

In one or more embodiments the modulating agent is used in a water in oil or oil in water emulsion. In one or more other embodiments the modulating agent is used in a unique waterless emulsion.

In certain embodiments the substance or residue may for example be acidic or basic and potentially alter pH in an emulsion environment or it may be one or more metal ions which may act as a potential catalyst in an emulsion environment.

In certain other embodiments the substance or residue may for example be acidic or basic and potentially alter an artificial pH in a waterless or substantially non aqueous environment or it may be one or more metal ions which may act as a potential catalyst in a waterless or substantially non aqueous environment.

In one or more embodiments the modulating agent is used to describe an agent which can affect pH in an aqueous solution. The agent can be any of the known buffering systems used in pharmaceutical or cosmetic formulations as would be appreciated by a man of the art. It can also be an organic acid, a carboxylic acid, a fatty acid an amino acid, an aromatic acid, an alpha or beta hydroxyl acid an organic base or a nitrogen containing compound.

In one or more further embodiments the modulating agent is used to describe an agent, which is a chelating or sequestering or complexing agent that is sufficiently soluble or functional in the solvent to enable it to “mop up” or “lock” metal ions.

In an embodiment modulating agent is used to describe an agent which can effect pH in an aqueous solution the term modulating agent more particularly means an acid or base or buffer system or combinations thereof, which is introduced into or is present in and acts to modulate the ionic or polar characteristics and any acidity or basesity balance of an emulsion carrier, composition, foamable carrier or foamable composition or resultant foam of the present invention.

In other embodiments modulating agent is used to describe an agent which can effect pH in an aqueous solution the term modulating agent more particularly means an acid or base or buffer system or combinations thereof, which is introduced into or is present in and acts to modulate the ionic or polar characteristics and any acidity or basesity balance of a waterless or substantially non aqueous carrier, composition, foamable carrier or foamable composition or resultant foam of the present invention.

The substance or residue can be introduced into the formulation from any one or more of the ingredients, some of which themselves may have acidic or basic properties. For example the polymer or solvent may contain basic residues in which case it may be desirable or beneficial to add an acid. Alternatively the surfactant may contain some acid residues in which case the addition of a base may be desirable and beneficial. In some cases more than one ingredient may contain residues which may ameliorate or compound their significance. For example if one ingredient provided weak acid residues and another stronger acid residues the pH in an emulsion environment (or artificial pH in a waterless environment) should be lower. In contrast if one residue was acid and the other basic the net effect in the formulation maybe significantly reduced. In some circumstances the active ingredient may favor an acidic pH or more significantly may need to be maintained at a certain acidic pH otherwise it may readily isomerize, chemically react or breakdown, in which case introducing acidic components such as an acidic polymer might be of help. In an embodiment of the present invention sufficient modulating agent is added to achieve a pH in which the active agent is preferably stable. In another embodiment of the present invention sufficient modulating agent is added to achieve an artificial pH in which the active agent is preferably stable.

The terms pH, pKa, and pKb, buffers and the like are used in classical measurements of an aqueous solution. Such measurements are artificial in a waterless environment. Nevertheless, reference to and description below of such terms are made for convenience and clarity, since such terms are well defined and understood with reference to aqueous solutions and further due to the lack of an appropriate uniform way of describing and identifying the artificial or virtual pH, pK etc in a waterless environment in relation to the present invention. Although predictions of artificial pH can be made using dilution techniques of measurements of waterless formulations diluted in water they are formulation sensitive and specific and have to be carefully calibrated with complex formulas.

Waterless medium can be polar and protic yet it does not conform to classical ionic behavior.

A buffer, as defined by Van Slyke [Van Slyke, J. Biol. Chem. 52, 525 (1922)], is “a substance which by its presence in solution increases the amount of acid or alkali that must be added to cause unit change in pH.”

A buffer solution is a solution of a definite pH made up in such a way that this pH alters only gradually with the addition of alkali or acid. Such a solution consists of a solution of a salt of the week acid in the presence of the three acid itself. The pH of the solution is determined by the dissociation equilibrium of the free acid.

An acid can be a strong acid or a weak acid. A strong acid is an acid, which is a virtually 100% ionized in solution. In contrast, a week acid is one which does not ionize fully. When it is dissolved in water. The lower the value for pKa, the stronger is the acid and likewise, the higher the value for pKa the weaker is the acid.

A base can be a strong base or a weak base. A strong base is something, which is fully ionic with 100% hydroxide ions. In contrast, a weak base is one which does not convert fully into hydroxide ions in solution. The lower the value for pKb, the stronger is the base and likewise, the higher the value for pKb the weaker is the base.

In one or more embodiments of the present invention the modulating agent comprises an organic compound.

In one or more preferred embodiments of the present invention the chelating agent is selected from the group consisting of ethylenediaminetetraacetic acid (EDTA), diethylenetriaminepentaacetic acid (DTPA), hydroxyethylenediaminetriacetic acid (HEDTA), nitrilotriacetic acid (NTA), O,O′-bis(2-aminoethyl)ethyleneglycol-N,N,N′,N′-tetraacetic acid (EGTA), trans-1,2-diaminocyclohexane-N,N,N′,N′-tetraacetic acid (CyPTA) or a pharmaceutically acceptable salt thereof (normally as a sodium salt), more preferably EDTA, HEDTA and their salts; most preferably EDTA and its salts.

In one or more embodiments of the present invention a preferred non limiting example of the chelating agent is EDTA. Typically, the chelating and sequestering agent is present in the composition at a level of up to about 5.0%, preferably 1.0 percent, by weight, of the composition.

In one or more embodiments of the present invention the modulating agent may also be a preservative or an antioxidant or an ionization agent. Any preservative, antioxidant or ionization agents suitable for pharmaceutical or cosmetic application may be used. Non limiting examples of antioxidants are tocopherol succinate, propyl galate, butylated hydroxy toluene and butyl hydroxy anisol. Ionization agents may be positive or may be negative depending on the environment and the active agent or composition that is to be protected. Ionization agents may for example act to protect or reduce sensitivity of active agents. Non limiting examples of positive ionization agents are benzyl conium chloride, and cetyl pyridium chloride. Non limiting examples of negative ionization agents are sodium lauryl sulphate, sodium lauryl lactylate and phospholipids.

### Heumectant

A heumectant is a substance that helps retain moisture and also prevents rapid evaporation. Non limiting examples are propylene glycol, propylene glycol derivatives, glycerin, hydrogenated starch hydrosylate, hydrogenated lanolin, lanolin wax, D manitol, sorbitol, sodium 2-pyrrolidone-5-carboxylate, sodium lactate, sodium PCA, soluble collagen, dibutyl phthalate, and gelatin. Other examples may be found in the Handbook of Pharmaceutical Additives published by Gower.

### Moisturizers

A moisturizer, is a substance that helps retain moisture or add back moisture to the skin. Examples are allantoin, petrolatum, urea, lactic acid, sodium PCV, glycerin, shea butter, caprylic/capric/stearic triglyceride, candelilla wax, propylene glycol, lanolin, hydrogenated oils, squalene, sodium hyaluronate and lysine PCA. Other examples may be found in the Handbook of Pharmaceutical Additives published by Gower.

Pharmaceutical compositions of the present invention may in one or more embodiments usefully comprise in addition a heumectant or a moisturizer or combinations thereof.

### Polar Solvent

A “polar solvent” is an organic solvent, typically soluble in both water and oil. Certain polar solvents, for example propylene glycol and glycerin, possess the beneficial property of a heumectant.

In one or more embodiments, the polar solvent is a heumectant.

In one or more embodiments, the polar solvent is a polyol. Polyols are organic substances that contain at least two hydroxy groups in their molecular structure.

In one or more embodiments, the polar solvent contains an diol (a compound that contains two hydroxy groups in its molecular structure), such as propylene glycol (e.g., 1,2-propylene glycol and 1,3-propylene glycol), butaneediol (e.g., 1,4-butaneediol), butaneediol (e.g., 1,3-butaneediol and 1,4-butenediol), butynediol, pentanediol (e.g., 1,5-pentanediol), hexanediol (e.g., 1,6-hexanediol), octanediol (e.g., 1,8-octanediol), neopentyl glycol, 2-methyl-1,3-propanediol, diethylene glycol, triethylene glycol, tetraethylene glycol, dipropylene glycol and dibutylene glycol.

In one or more embodiments, the polar solvent contains a triol (a compound that contains three hydroxy groups in its molecular structure), such as glycerin and 1,2,6-Hexanetriol.

Other non-limiting examples of polar solvents include pyrrolidones, (such as N-methyl-2-pyrrolidone and 1-methyl-2-pyrrolidinone), dimethyl isosorbide, 1,2,6-hexapetriol, dimethyl sulfoxide (DMSO), ethyl proxitol, dimethylacetamide (DMAc) and alpha hydroxy acids, such as lactic acid and glycolic acid.

According to still other embodiments, the polar solvent is a polyethylene glycol (PEG) or PEG derivative that is liquid at ambient temperature, including PEG200 (MW (molecular weight) about 190-210 kD), PEG300 (MW about 285-315 kD), PEG400 (MW about 380-420 kD), PEG600 (MW about 570-630 kD) and higher MW PEGs such as PEG 4000, PEG 6000 and PEG 10000 and mixtures thereof.

Polar solvents are known to enhance the penetration of active agent into the skin and through the skin, and therefore, their inclusion in the composition of the present invention can be desirable, despite their undesirable skin drying and irritation potential. There is at one level a commonality between the different polar solvents and their penetration enhancement properties. Lower molecular weight alcohols can sometimes be more potent as a solvent, for example by extracting lipids from the skin layers more effectively, which characteristic can adversely affect the skin structure and cause dryness and irritation. Therefore the selection of lower molecular weight alcohols is ideally avoided.

### Skin Penetration Enhancer

A “skin penetration enhancer”, also termed herein “penetration enhancer,” is an organic solvent, typically soluble in both water and oil. Examples of penetration enhancer include polyols, such as glycerol (glycerin), propylene glycol, hexylene glycol, diethylene glycol, propylene glycol n-alkanols, terpenes, di-terpenes, tri-terpenes, terpen-ols, limonene, terpene-ol, 1-menthol, dioxolane, ethylene glycol, hexylene glycol, other glycols, sulfoxides, such as dimethylsulfoxide (DMSO), dimethylformanide, methyl dodecyl sulfoxide, dimethylacetamide, dimethylisosorbide, monooleate of ethoxylated glycerides (with 8 to 10 ethylene oxide units), azone (1-dodecylazacycloheptan-2-one), 2-(n-nonyl)-1,3-dioxolane, esters, such as isopropyl myristate/palmitate, ethyl acetate, butyl acetate, methyl proprionate, capric/caprylic triglycerides, octylmyristate, dodecyl-myristate; myristyl alcohol, lauryl alcohol, lauric acid, lauryl lactate ketones; amides, such as acetamide oleates such as triolein; various alkanoic acids such as caprylic acid; lactam compounds, such as azone; alkanols, such as dialkylamino acetates, and admixtures thereof.

According to one or more embodiments, the penetration enhancer is a polyethylene glycol (PEG) or PEG derivative that is liquid at ambient temperature

### Potent Solvent

In one or more embodiments of the present invention, the foamable composition includes a potent solvent, in addition to or in place of one of the hydrophobic solvents, polar solvents or emollients of the composition. A potent solvent is a solvent other than mineral oil that solubilizes a specific active agent substantially better than a hydrocarbon solvent such as mineral oil or petrolatum. For example, a potent solvent solubilizes the active agent 5 fold better than a hydrocarbon solvent; or even solubilizes the active agent 10-fold better than a hydrocarbon solvent.

In one or more embodiments of the present invention, the composition includes at least one active agent in a therapeutically effective concentration; and at least one potent solvent in a sufficient amount to substantially solubilize the at least one active agent in the composition. The term “substantially soluble” means that at least 95% of the active agent has been solubilized, i.e., 5% or less of the active agent is present in a solid state. In one or more embodiments, the concentration of the at least one potent solvent is more than about 40% of the at least one solvent of the composition of the present invention; or even more than about 60%.

Non-limiting examples of pairs of active agent and potent solvent include:

Betamethasone valerate: Practically insoluble in mineral oil (<0.01%); soluble more than 1% in glycofurol; Hydrocortisone butyrate: Practically insoluble in mineral oil (<0.01%); soluble more than 1% in glycofurol; Metronidazole: Practically insoluble in mineral oil (<0.01%); soluble more than 1% in dimethyl isosrbide; Ketoconazole: Practically insoluble in mineral oil (<0.01%); soluble more than 1% in glycofurol, propylene glycol and dimethyl isosrbide; Mupirocin: Practically insoluble in mineral oil (<0.01%); soluble more than 1% in glycofurol, hexylene glycol, dimethyl isosorbide, propylene glycol and polyethylene glycol 400 (PEG 400); Meloxicam, a nonsteroidal anti-inflammatory agent: Practically insoluble in mineral oil (<0.001%); soluble in propylene glycol: 0.3 mg/mL; and in PEG 400: 3.7 mg/mL; and Progesterone: Practically insoluble in mineral oil (<0.001%); soluble in PEG 400: 15.3 mg/mL.

A non-limiting exemplary list of solvents that can be considered as potent solvents includes polyethylene glycol, propylene glycol, hexylene glycol, butaneediols and isomers thereof, glycerol, benzyl alcohol, DMSO, ethyl oleate, ethyl caprylate, diisopropyl adipate, dimethylacetamide, N-methylpyrrolidone, N-hydroxyethylpyrrolidone, polyvinylpyrrolidone, isosorbide derivatives, such as dimethyl isosorbide, glycofurol and ethoxydiglycol (transcutol) and laurocapram.

The use of a potent solvent in a foam composition provides an improved method of delivering poorly soluble therapeutic agents to a target area. It is known that low drug solubility results in poor bioavailability, leading to decreased effectiveness of treatment. Foam compositions of the present invention, for which the solvent includes a potent solvent, increase the levels of the active agent in solution and thus, provide high delivery and improved therapy.

Potent solvents, as defined herein, are usually liquid. Formulations comprising potent solvents and active agents are generally disadvantageous as therapeutics, since their usage involves unwanted dripping and inconvenient method of application; resulting in inadequate dosing. Surprisingly, the foams of the present invention, which are drip-free, provide a superior vehicle for such active agents, enabling convenient usage and accurate effective dosing.

In one or more embodiments of the present invention the present invention the foamable pharmaceutical composition may additionally include a mixture of two or more of the solvents selected from the group of hydrophobic solvents, silicone oils, emollients, polar solvents and potent solvents in an appropriate proportion as would be appreciated to a person skilled in the art.

In one or more embodiments of the present invention, the PPG alkyl ether may act as a potent solvent

### Composition and Foam Physical Characteristics and Advantages

A pharmaceutical or cosmetic composition manufactured using the foamable carrier of the present invention is very easy to use. When applied onto the afflicted body surface of mammals, i.e., humans or animals, it is in a foam state, allowing free application without spillage. Upon further application of a mechanical force, e.g., by rubbing the composition onto the body surface, it freely spreads on the surface and is rapidly absorbed.

The foamable composition of the present invention is stable, having an acceptable shelf-life of at least one year, or preferably, at least two years at ambient temperature, as revealed in accelerated stability tests. The foamable compositions according to the present invention are stable. Following accelerated stability studies, they demonstrate desirable texture; they form fine bubble structures that do not break immediately upon contact with a surface, spread easily on the treated area and absorb quickly.

The composition should also be free flowing, to allow it to flow through the aperture of the container, e.g., and aerosol container, and create an acceptable foam.

Foam quality can be graded as follows:

Grade E (excellent): very rich and creamy in appearance, does not show any bubble structure or shows a very fine (small) bubble structure; does not rapidly become dull; upon spreading on the skin, the foam retains the creaminess property and does not appear watery.

Grade G (good): rich and creamy in appearance, very small bubble size, “dulls” more rapidly than an excellent foam, retains creaminess upon spreading on the skin, and does not become watery.

Grade FG (fairly good): a moderate amount of creaminess noticeable, bubble structure is noticeable; upon spreading on the skin the product dulls rapidly and becomes somewhat lower in apparent viscosity.

Grade F (fair): very little creaminess noticeable, larger bubble structure than a “fairly good” foam, upon spreading on the skin it becomes thin in appearance and watery.

Grade P (poor): no creaminess noticeable, large bubble structure, and when spread on the skin it becomes very thin and watery in appearance.

Grade VP (very poor): dry foam, large very dull bubbles, difficult to spread on the skin.

Topically administrable foams are typically of quality grade E or G, when released from the aerosol container. Smaller bubbles are indicative of more stable foam, which does not collapse spontaneously immediately upon discharge from the container. The finer foam structure looks and feels smoother, thus increasing its usability and appeal.

As further aspect of the foam is breakability. The breakable foam is thermally stable, yet breaks under sheer force. Sheer-force breakability of the foam is clearly advantageous over thermally induced breakability. Thermally sensitive foams immediately collapse upon exposure to skin temperature and, therefore, cannot be applied on the hand and afterwards delivered to the afflicted area.

Another property of the foam is specific gravity, as measured upon release from the aerosol can. Typically, foams have specific gravity of less than 0.12 g/mL; or less than 0.10 g/mL; or less than 0.08 g/mL, depending on their composition and on the propellant concentration.

### Pharmaceutical Composition

The foamable carrier of the present invention is an ideal vehicle for active pharmaceutical ingredients and active cosmetic ingredients. In the context of the present invention, active pharmaceutical ingredients and active cosmetic ingredients are collectively termed “active agent” or “active agents”.

Suitable active agents include but are not limited to active herbal extracts, acaricides, age spot and keratose removing agents, allergen, analgesics, local anesthetics, antiacne agents, antiallergic agents, antiaging agents, antibacterials, antibiotics, antiburn agents, anticancer agents, antidandruff agents, antidepressants, antidermatitis agents, antiedemics, antihistamines, antihelminths, antihyperkeratolyte agents, antiinflammatory agents, antiirritants, antilipemics, antimicrobials, antimycotics, antiproliferative agents, antioxidants, anti-wrinkle agents, antipruritics, antipsoriatic agents, antirosacea agents antiseborrheic agents, antiseptic, antiswelling agents, antiviral agents, antiyeast agents, astringents, topical cardiovascular agents, chemotherapeutic agents, corticosteroids, dicarboxylic acids, disinfectants, fungicides, hair growth regulators, hormones, hydroxy acids, immunosuppressants, immunoregulating agents, insecticides, insect repellents, keratolytic agents, lactams, metals, metal oxides, mitocides, neuropeptides, non-steroidal anti-inflammatory agents, oxidizing agents, pediculicides, photodynamic therapy agents, retinoids, sanatives, scabicides, self tanning agents, skin whitening agents, asoconstrictors, vasodilators, vitamins, vitamin D derivatives, wound healing agents, wart removers, an anti-infective, an antibiotic, an antibacterial agent, an antifungal agent, an antiviral agent, an antiparasitic agent, an immunosuppressive agent, an immunomodulator, an immunoregulating agent, a hormonal agent, a steroid; vitamin A, a vitamin A derivative, vitamin B, a vitamin B derivative, vitamin C, a vitamin C derivative, vitamin D, a vitamin D derivative; vitamin E, a vitamin E derivative, vitamin F, a vitamin F derivative, vitamin K, a vitamin K derivative, a wound healing agent, a disinfectant, an anesthetic, an antiallergic agent, an alpha hydroxyl acid, lactic acid, glycolic acid, a beta-hydroxy acid, a protein, a peptide, a neuropeptide, a allergen, an immunogenic substance, a haptene, an oxidizing agent, an antioxidant, a dicarboxylic acid, azelaic acid, sebacic acid, adipic acid, fumaric acid, a retinoid, an antiproliferative agent, an anticancer agent, a photodynamic therapy agent, benzoyl chloride, calcium hypochlorite, magnesium hypochlorite, an anti-wrinkle agent, a radical scavenger, a metal, silver, a metal oxide, titanium dioxide, zinc oxide, zirconium oxide, iron oxide, silicone oxide, talc, carbon, an anti wrinkle agent, a skin whitening agent, a skin protective agent, a masking agent, an anti-wart agent, a refatting agent, a lubricating agent and mixtures thereof. As is known to one skilled in the art, in some instances a specific active agent may have more than one activity, function or effect.

### Fields of Applications

The foamable carrier of the present invention is suitable for treating any inflicted surface. In one or more embodiments, foamable carrier is suitable for administration to the skin, a body surface, a body cavity or mucosal surface, e.g., the cavity and/or the mucosa of the nose, mouth, eye, ear, respiratory system, vagina or rectum (severally and interchangeably termed herein “target site”).

By selecting a suitable active agent, or a combination of at least two active agents, the foamable composition of the present invention is useful in treating an animal or a human patient having any one of a variety of dermatological disorders, including dermatological pain, dermatological inflammation, acne, acne vulgaris, inflammatory acne, non-inflammatory acne, acne fulminans, nodular papulopustular acne, acne conglobata, dermatitis, bacterial skin infections, fungal skin infections, viral skin infections, parasitic skin infections, skin neoplasia, skin neoplasms, pruritis, cellulitis, acute lymphangitis, lymphadenitis, erysipelas, cutaneous abscesses, necrotizing subcutaneous infections, scalded skin syndrome, folliculitis, furuncles, hidradenitis suppurativa, carbuncles, paronychial infections, rashes, erythrasma, impetigo, eethyma, yeast skin infections, warts, molluscum contagiosum, trauma or injury to the skin, post-operative or post-surgical skin conditions, scabies, pediculosis, creeping eruption, eczemas, psoriasis, pityriasis rosea, lichen planus, pityriasis rubra pilaris, edematous, erythema multiforme, erythema nodosum, granuloma annulare, epidermal necrolysis, sunburn, photosensitivity, pemphigus, bullous pemphigoid, dermatitis herpetiformis, keratosis pilaris, callouses, corns, ichthyosis, skin ulcers, ischemic necrosis, miliaria, hyperhidrosis, moles, Kaposi's sarcoma, melanoma, malignant melanoma, basal cell carcinoma, squamous cell carcinoma, poison ivy, poison oak, contact dermatitis, atopic dermatitis, rosacea, purpura, moniliasis, candidiasis, baldness, alopecia, Behcet's syndrome, cholesteatoma, Dercum disease, ectodermal dysplasia, gustatory sweating, nail patella syndrome, lupus, hives, hair loss, Hailey-Hailey disease, chemical or thermal skin burns, scleroderma, aging skin, wrinkles, sun spots, necrotizing fasciitis, necrotizing myositis, gangrene, scarring, and vitiligo.

Likewise, the foamable composition of the present invention is suitable for treating a disorder of a body cavity or mucosal surface, e.g., the mucosa of the nose, mouth, eye, ear, respiratory system, vagina or rectum. Non limiting examples of such conditions include chlamydia infection, gonorrhea infection, hepatitis B, herpes, HIV/AIDS, human papillomavirus (HPV), genital warts, bacterial vaginosis, candidiasis, chancroid, granuloma Inguinale, lymphogranuloma venereum, mucopurulent cervicitis (MPC), molluscum contagiosum, nongonococcal urethritis (NGU), trichomoniasis, vulvar disorders, vulvodynia, vulvar pain, yeast infection, vulvar dystrophy, vulvar intraepithelial neoplasia (VIN), contact dermatitis, pelvic inflammation, endometritis, salpingitis, oophoritis, genital cancer, cancer of the cervix, cancer of the vulva, cancer of the vagina, vaginal dryness, dyspareunia, anal and rectal disease, anal abscess/fistula, anal cancer, anal fissure, anal warts, Crohn's disease, hemorrhoids, anal itch, pruritus ani, fecal incontinence, constipation, polyps of the colon and rectum.

In an embodiment of the present invention, the composition is useful for the treatment of an infection. In one or more embodiments, the composition is suitable for the treatment of an infection, selected from the group of a bacterial infection, a fungal infection, a yeast infection, a viral infection and a parasitic infection.

In an embodiment of the present invention, the composition is useful for the treatment of wound, ulcer and burn.

In an embodiment of the present invention, the target site is selected from the group consisting of the skin, a body cavity, a mucosal surface, the nose, the mouth, the eye, the ear canal, the respiratory system, the vagina and the rectum.

The composition of the present invention is also suitable for administering a hormone to the skin or to a mucosal membrane or to a body cavity, in order to deliver the hormone into the tissue of the target organ, in any disorder that responds to treatment with a hormone.

In an embodiment of the present invention, the target site is selected from the group consisting of the skin, a body cavity, a mucosal surface, the nose, the mouth, the eye, the ear canal, the respiratory system, the vagina and the rectum. In an embodiment of the present invention, the disorder is selected from the group consisting of dermatological pain, dermatological inflammation, acne, acne vulgaris, inflammatory acne, non-inflammatory acne, acne fulminans, nodular papulopustular acne, acne conglobata, dermatitis, bacterial skin infections, fungal skin infections, viral skin infections, parasitic skin infections, skin neoplasia, skin neoplasms, pruritis, cellulitis, acute lymphangitis, lymphadenitis, erysipelas, cutaneous abscesses, necrotizing subcutaneous infections, scalded skin syndrome, folliculitis, furuncles, hidradenitis suppurativa, carbuncles, paronychial infections, rashes, erythrasma, impetigo, eethyma, yeast skin infections, warts, molluscum contagiosum, trauma or injury to the skin, post-operative or post-surgical skin conditions, scabies, pediculosis, creeping eruption, eczemas, psoriasis, pityriasis rosea, lichen planus, pityriasis rubra pilaris, edematous, erythema multiforme, erythema nodosum, granuloma annulare, epidermal necrolysis, sunburn, photosensitivity, pemphigus, bullous pemphigoid, dermatitis herpetiformis, keratosis pilaris, callouses, corns, ichthyosis, skin ulcers, ischemic necrosis, miliaria, hyperhidrosis, moles, Kaposi's sarcoma, melanoma, malignant melanoma, basal cell carcinoma, squamous cell carcinoma, poison ivy, poison oak, contact dermatitis, atopic dermatitis, rosacea, purpura, moniliasis, candidiasis, baldness, alopecia, Behcet's syndrome, cholesteatoma, Dercum disease, ectodermal dysplasia, gustatory sweating, nail patella syndrome, lupus, hives, hair loss, Hailey-Hailey disease, chemical or thermal skin burns, scleroderma, aging skin, wrinkles, sun spots, necrotizing fasciitis, necrotizing myositis, gangrene, scarring, and vitiligo, chlamydia infection, gonorrhea infection, hepatitis B, herpes, HIV/AIDS, human papillomavirus (HPV), genital warts, bacterial vaginosis, candidiasis, chancroid, granuloma Inguinale, lymphogranuloma venereum, mucopurulent cervicitis (MPC), molluscum contagiosum, nongonococcal urethritis (NGU), trichomoniasis, vulvar disorders, vulvodynia, vulvar pain, yeast infection, vulvar dystrophy, vulvar intraepithelial neoplasia (VIN), contact dermatitis, pelvic inflammation, endometritis, salpingitis, oophoritis, genital cancer, cancer of the cervix, cancer of the vulva, cancer of the vagina, vaginal dryness, dyspareunia, anal and rectal disease, anal abscess/fistula, anal cancer, anal fissure, anal warts, Crohn's disease, hemorrhoids, anal itch, pruritus ani, fecal incontinence, constipation, polyps of the colon and rectum; and wherein the active agent is suitable for treating said disorder.

In one embodiment of the present invention, the disorder is psoriasis; the active agent is a vitamin D derivative, given at a concentration between about 0.001% and about 0.02% by weight.

In an embodiment of the present invention, the active agent is selected from the group comprising of: Hydrocortisone acetate, Betamethasone valerate, Clobetasol proprionate, Acyclovir, Ciclopirox, Clindamycin, Azelaic acid, Metronidazol, Diclofenac, Tacrolimus, Caffeine, Clotrimazole, Lidocaine base, Terbinafine HCL, Gentamycin, Dexpanthenol, Urea, Ammonium lactate, Povidone-iodine and Permethrine.

In one embodiment of the present invention, the active agent is a permethrin. Preferably, at a concentration between about 1% and about 8% by weight.

The following examples further exemplify the PPG foamable pharmaceutical carriers, pharmaceutical compositions thereof, methods for preparing the same, and therapeutic uses of the compositions. The examples are for the purposes of illustration only and are not intended to be limiting of the invention. Many variations may be carried out by one of ordinary skill in the art and are contemplated within the full scope of the present invention.

In one embodiment of the present invention, the foamable compositions and foams are suitable for use in treating, ameliorating, reducing or preventing a dermatological, cosmetic or mucosal disorder. More particularly, they are suitable for use where such disorders would otherwise be less responsive when treated with one agent alone.

### Methodology

A general procedure for preparing foamable compositions is set out in WO 2004/037225, which is incorporated herein by reference.

### Emulsion Foam

1. Mix oily phase ingredients and heat to 75° C. to melt all ingredients and obtain homogeneous mixture.
2. Mix polymers in water with heating or cooling as appropriate for specific polymer.
3. Add all other water soluble ingredients to water-polymer solution and heat to 75° C.
4. Add slowly internal phase to external phase at 75° C. under vigorous mixing and homogenize to obtain fine emulsion. Alternatively the external phase is added slowly to the internal phase.
5. Cool to below 40° C. and add sensitive ingredients with mild mixing.
6. Cool to room temperature.

### Waterless Foam

1. Dissolve the polymers in the main solvent with heating or cooling as appropriate for specific polymer. Add the all other ingredients and heat to 75° C. to melt and dissolve the various ingredients.
2. Cool to below 40° C. and add sensitive ingredients with mild mixing.
3. Cool to room temperature.

### Oily Waterless Foam

1. Mix all ingredients excluding polymers and heat to 75° C. to melt and dissolve and obtain homogeneous mixture.
2. Mix well and cool to below 40° C. and add the polymers and sensitive ingredients with moderate mixing.
3. Cool to room temperature.
   Oily Foam with Phospholipids and/or Water
1. Swell the phospholipids in the main oily solvent under mixing for at least 20 minutes until uniform suspension is obtained.
2. Add all other ingredients excluding polymers and heat to 75° C. to melt and dissolve and obtain homogeneous mixture.
3. Mix well and cool to below 40° C. and add the polymers and sensitive ingredients with moderate mixing.
4. Cool to room temperature.
5. In case of polymers dissolved in water or organic solvent, dissolve the polymers in the solvent with heating or cooling as appropriate for specific polymer and add to the oily mixture under vigorous mixing at ˜40° C.

### Canisters Filling and Crimping

Each aerosol canister is filled with PFF and crimped with valve using vacuum crimping machine.

### Pressurizing

Propellant Filling
Pressurizing is carried out using a hydrocarbon gas or gas mixture Canisters are filled and then warmed for 30 sec in a warm bath at 50° C. and well shaken immediately thereafter.

Closure Integrity Test.
Each pressurized canister is subjected to bubble and crimping integrity testing by immersing the canister in a 60° C. water bath for 2 minutes. Canisters are observed for leakage as determined by the generation of bubbles. Canisters releasing bubbles are rejected.

### Tests

By way of non limiting example the objectives of hardness, collapse time and FTC stability tests are briefly set out below as would be appreciated by a person of the art.

Hardness

LFRA100 instrument is used to characterize hardness. A probe is inserted into the test material. The resistance of the material to compression is measured by a calibrated load cell and reported in units of grams on the texture analyzer instrument display. Preferably at least three repeat tests are made. The textural characteristics of a dispensed foam can effect the degree of dermal penetration, efficacy, spreadability and acceptability to the user. The results can also be looked at as an indicator of softness. Note: the foam sample is dispensed into an aluminum sample holder and filled to the top of the holder.

Collapse Time

Collapse time (CT) is examined by dispensing a given quantity of foam and photographing sequentially its appearance with time during incubation at 36° C. It is useful for evaluating foam products, which maintain structural stability at skin temperature for at least 1 min.

Viscosity

Viscosity is measured with Brookfield LVDV-II+PRO with spindle SC₄₋₂₅ at ambient temperature and 10, 5 and 1 RPM. Viscosity is usually measured at 10 RPM. However, at about the apparent upper limit for the spindle of ˜>50,000 CP, the viscosity at 1 RPM may be measured, although the figures are of a higher magnitude.

FTC (Freeze Thaw Cycles)

To check the foam appearance under extreme conditions of repeated cycles of cooling, heating, (first cycle) cooling, heating (second cycle) etc., commencing with −100° C. (24 hours) followed by +400° C. (24 hours) measuring the appearance and again repeating the cycle for up to three times.

Creaming by centrifugation:

### 1. Principle of Test

The centrifugation used in this procedure serves as a stress condition simulating the aging of the liquid dispersion under investigation. Under these conditions, the centrifugal force applied facilitates the coalescence of dispersed globules or sedimentation of dispersed solids, resulting in loss of the desired properties of the formulated dispersion.

### 2. Procedure

2.1. Following preparation of the experimental formulation/s, allow to stand at room temperature for ≧24 h.
2.2. Handle pentane in the chemical hood. Add to each experimental formulation in a 20-mL glass vial a quantity of pentane equivalent to the specified quantity of propellant for that formulation, mix and allow formulation to stand for at least 1 h and not more than 24 h.
2.3. Transfer each mixture to 1.5 mL microtubes. Tap each microtube on the table surface to remove entrapped air bubbles.
2.4. Place visually balanced microtubes in the centrifuge rotor and operate the centrifuge at 3,000 rpm for 10 min or at 1,000 rpm for 10 min.

### Stock Compositions

Non-limiting examples of how stock solutions are made up with and without API. Other stock solutions may be made using the same methodology by simply varying adding or omitting ingredients as would be appreciated by one of the ordinary skills in the art. Reference to propellant in the examples is to propane, isobutene, butane mixtures although other proellants can be used as would be appreciated by a man of the art.

### EXAMPLES

The invention is described with reference to the following examples. This invention is not limited to these examples and experiments. Many variations will suggest themselves and are within the full intended scope of the appended claims.

### Example 1

### Foamable Oil in Water Emulsion Vehicle Compositions, Containing High Concentrations (More than 15% and Up to 60%) PPG Alky Ether

PART A - 35% PPG with solid and liquid surfactants

| Ingredients | % |
|---|---|
| PPG 15 stearyl ether | 35.00 |
| Glyceryl Monostearate | 0.49 |
| Sorbitane Stearate | 0.65 |
| Stearyl Alcohol | 0.92 |
| Steareth-21 | 2.17 |
| PEG-40 Stearate | 2.83 |
| Methocel A4M | 0.33 |
| Xanthan gum | 0.28 |
| Polysorbate 80 | 0.98 |
| Water purified | 56.35 |
| Total | 100.00 |
| Propellant (propane; | 8.00 |
| isobutane; butane) | |
| Foam Appearance | |
| Quality | G-E |
| Color | white |
| Odor | no odor |
| Shakability | good |

G-E = Good to Excellent
Notes
The liquefied or gas propellant can be added at a concentration of
about 3% to about 25%.

PART B - 35% PPG with liquid surfactants

| Ingredients | % |
|---|---|
| PPG 15 Stearyl ether | 35.00 |
| Laureth 4 | 2.00 |
| Span 80 | 2.00 |
| Methocel A4M | 0.33 |
| Xanthan gum | 0.28 |
| Polysorbate 80 | 2.00 |
| Purified water | 58.39 |
| Total | 100.00 |
| Propellant (propane; | 8.00 |
| isobutane; butane) | |
| Foam Appearance | |
| Quality | G-E |
| Color | White |
| Odor | No odor |
| Shakability | Good |

G-E = Good to Excellent
Notes:
The liquefied or gas propellant can be added at a concentration of about
3% to about 25%.
An aqueous formulation containing only liquid surfactants was
successfully prepared and confirms that good foam can be prepared
even with only liquid surfactants and PPG.

PART C - 20%, 40% and 45% PPG with solid surfactants and
with/without a polymeric agent.

| Ingredients | % | % | % | % |
|---|---|---|---|---|
| PPG 15 Stearyl ether | 20.00 | 40.00 | 40.00 | 45.00 |
| CarboxyMethylCellulose | 0.50 | 0.50 | — | — |
| (CMC) | | | | |
| Steareth-2 | 4.00 | 7.00 | 7.00 | 7.00 |
| Steareth-21 | 1.00 | 3.00 | 3.00 | 3.00 |
| Purified water | 74.50 | 49.50 | 50.00 | 45.00 |
| Total | 100.00 | 100.00 | 100.00 | 100.00 |
| Propellant (propane; | 8.00 | 8.00 | 8.00 | 8.00 |
| isobutane; butane) | | | | |
| Foam Appearance | | | | |
| Quality | G-E | G-E | G-E | G-E |
| Color | White | White | White | White |
| Odor | No odor | No odor | No odor | No odor |
| Shakability | Good | Good | Good | Good |

G-E = Good to Excellent
Notes:
The liquefied or gas propellant can be added at a concentration of
about 3% to about 25%
An aqueous formulation containing only solid surfactants was successfully
prepared and confirms that good foam can be prepared even with only
solid surfactants and PPG.
Polymeric agents are usually helpful and often essential in improving
the foam quality. Unexpectedly, formulations with higher levels of PPG
containg poymeric agent did not form good foams. It was discovered
that when polymeric agents were eliminated it was not only possible to
make good foams but the quality can be excellent Thus, for PPG foam
omission of polymeric agent can be an unexpected advantage.at least at
higher levels of PPG. The polymers Klucel EF and Carbomer 934 are
insoluble in PPG 15 stearyl ether

PART D - About 40% PPG with single solid surfactants and
with a polymeric agent.

| Ingredients | % | % | % | % |
|---|---|---|---|---|
| PPG 15 Stearyl ether | 40.00 | 44.00 | 43.50 | 43.50 |
| CarboxyMethylCellulose | 1.00 | 1.00 | 0.50 | 0.50 |
| (CMC) | | | | |
| GMS | | | | 6.00 |
| Ceteareth 20 | 9.00 | 5.00 | 6.00 | |
| Purified water | 50.00 | 50.00 | 50.00 | 50.00 |
| Total | 100.00 | 100.00 | 100.00 | 100.00 |
| Propellant (propane; | 8.00 | 8.00 | 8.00 | 8.00 |
| isobutane; butane) | | | | |
| Foam Appearance | | | | |
| Quality | G-E | G-E | G-E | G-E |
| Color | White | White | White | White |
| Odor | No odor | No odor | No odor | No odor |
| Shakability | Good | Good | Good | Good |

G-E = Good to Excellent
Notes:
The liquefied or gas propellant can be added at a concentration of about
3% to about 25%.
Good quality foams can be prepared from aqueous compositions
containing 45% PPG with a single surfactant.

PART E - 60% PPG with one or more surfactants and with/without
a polymeric agent.

| Ingredients | A % | B % |
|---|---|---|
| PPG 15 Stearyl ether | 60.00 | 60.00 |
| CarboxyMethylCellulose | — | 0.50 |
| (CMC) | | |
| GMS | | 6.00 |
| Steareth-2 | 7.00 | |
| Steareth-21 | 3.00 | |
| Purified water | 30.00 | 33.50 |
| Total | 100.00 | 100.00 |
| Propellant (propane; | 8.00 | 8.00 |
| isobutane; butane) | | |
| Foam Appearance | | |
| Quality | G-E | G-E |
| Color | White | White |
| Odor | No odor | No odor |
| Shakability | Good | Good |
| Density | NM | 0.154 |
| Creaming after 3000 rpm | | Homogenous |
| centrifugation | | |
| Collapse time (seconds) | | >300 |
| Expansion time | | >300 |
| (seconds) | | |
| Visual inspection (GB) | | Homogenous |
| Hardness (g) | | 14.31 |
| Viscosity (cP) | | 10607 |

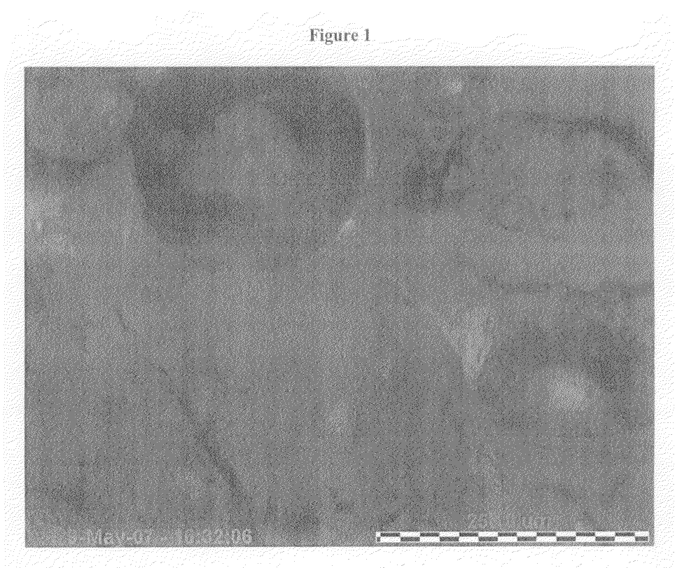

G-E = Good to Excellent
NM = Not Measured
Notes:
The liquefied or gas propellant can be added at a concentration of about
3% to about 25%.
Good foams can be prepared from aqueous compositions containing
60% PPG and only about 30% of water.
An aqueous oil in water formulation containing GMS (Surfactant) and
CMC (polymer) produced an good foam as did a similar formulation with
other surfactants but no polymer. Thus, a foam containing very high
amounts of PPG can be prepared with or without polymer.
Formulation B was examined under a polarized microscope at X1000
(see FIG. 1) and appeared to be a mixture of various ordered structures
and solids in suspension, whereas the GMS and water appeared to be
solubilized in the PPG main solvent and form solids and ordered
structures, such as liquid crystals.
Attempts to raise the PPG content of aqueous formulations to above 60%
resulted in segregation of the surfactant particles. An attempt at reducing
the surfactants and at not using surfactants appeared to be successful in
avoiding segregation, but on the other hand did not result in a stable or
satisfactory foam.

PART F - Preliminary Comparison of Usability of 60% PPG foam with
Petrolatum and Baby Mousik

| | Ease of | skin | Shiny | |
|---|---|---|---|---|
| | application | absorbtion | Look | comfort |
| Average grade | | | | |
| immediately after | | | | |
| application on skin | | | | |
| Tested: Petrolatum | 1.7 | 1.6 | 1.8 | 1.8 |
| Tested: Example 1 | 2.4 | 1.6 | 1 | 1.6 |
| Part E, Formula B | | | | |
| Tested: Baby | 2.4 | 2.8 | 2.8 | 2.8 |
| Mussik | | | | |
| Average grade after | | | | |
| 15 minutes from | | | | |
| application on skin | | | | |
| Tested: petrolatum | 2 | 1.6 | 1.6 | 2.1 |
| Example 1 Part E, | 2.4 | 2.4 | 2.6 | 2.4 |
| Formula B | | | | |
| Tested: Baby | 2.4 | 3 | 3 | 3 |
| Mussik | | | | |

0: worst→ 3 excellent
Notes:
Formulation B was considered by the testers to be generally comparible
to a commercially used baby lotionn (oinment), in particular after 15
minutes from application time.

### Example 2

### Waterless Foamable Vehicle Compositions, Containing High Concentrations (Up to 83%) PPG Alky Ether

PART A - 74.5% TO 83% PPG and Cocogycerides

| Ingredients | A % | B % | C % | D % | E % |
|---|---|---|---|---|---|
| PPG 15 Stearyl | 79.50 | 83.00 | 74.50 | 74.50 | 77.50 |
| ether | | | | | |
| GMS | 8.50 | 7.00 | 8.50 | 8.50 | 8.50 |
| Stearyl alcohol | 6.00 | 5.00 | 6.00 | 6.00 | 6.00 |
| Cocoglycerides | 6.00 | 5.00 | 6.00 | 6.00 | 6.00 |
| Propylene glycol | | | 5.00 | | |
| PEG 400 | | | | 5.00 | |
| Cetyl methicone | | | | | 2.00 |
| total: | 100.00 | 100.00 | 100.00 | 100.00 | 100.00 |
| Propellant | 8.00 | 8.00 | 8.00 | 8.00 | 8.00 |
| (propane; | | | | | |
| isobutane; | | | | | |
| butane) | | | | | |
| Foam | | | | | |
| Appearance | | | | | |
| Quality | G-E | G | G- | G- | G- |
| Color | White | White | White | White | White |
| Odor | No odor | No odor | No | No | No odor |
| | | | odor | odor | |
| Shakability | Good | Good | Good | Good | Good |

| | | |
|---|---|---|
| Density | 0.168 | Not Measured |
| Creaming after | Homogenous | |
| 3000 rpm | | |
| centrifugation | | |
| Creaming after | 90% | |
| 10000 rpm | Separation | |
| centrifugation | | |
| Expansion time | >300 | |
| (seconds) | | |
| Visual inspection | Homogenous | |
| (GB) | | |
| Hardness (g) | 9.76 | |
| Viscosity (cP) | 16117 | |

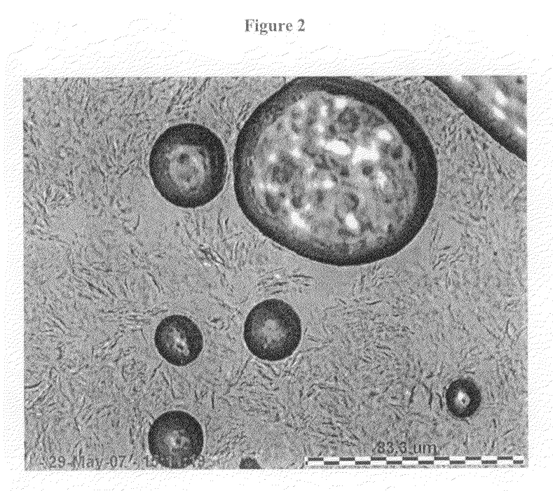

G-E = Good to Excellent
Notes:
The liquefied or gas propellant can be added at a concentration of about
3% to about 25%.
Good foams can be prepared from substantially waterless compositions
containing over 74% PPG.
Thus, a waterless foam containing very high amounts of PPG can be
prepared, wherein PPG is the main solvent, with a foam adjuvant and
solid triglycerides (cocoglycerides) and a stabilizing surfactant (GMS
glyceryl stearate)
Formulation A was examined under a polarized microscope at X600
(see FIG. 2) and appeared to be a solid suspension of cocoglycerides
and stearyl alcohol mixture solids suspended in the main solvent PPG.
The solid particles are typically uniform rods of 5 to 10 microns length.
Thus, PPG and cococlycerides have been discoved to have a
unique microscopic sructure.

PART B - Preliminary Comparison of Usability of 60%
PPG foam with Petrolatum and Baby Mousik

| | Ease of | skin | Shiny | |
|---|---|---|---|---|
| | application | absorbtion | Look | comfort |
| Average grade | | | | |
| immediately after | | | | |
| application on skin | | | | |
| Tested: petrolatum | 1.4 | 0.6 | 0.4 | 1.3 |
| Tested: Baby | 2.4 | 1.8 | 1.2 | 1.8 |
| Mussik | | | | |
| Tested: Example 2, | 2.2 | 1.6 | 1.4 | 2 |
| Part 1, Formula A | | | | |
| Average grade after | | | | |
| 15 minutes from | | | | |
| application on skin | | | | |
| Tested: petrolatum | 1.3 | 0.5 | 0.5 | 0.6 |
| Tested: Baby | 2.4 | 2.4 | 2.6 | 2.4 |
| Mussik | | | | |
| Tested: Example 2, | 2.2 | 2.6 | 2.8 | 2.4 |
| Part 1, Formula A | | | | |

0: worst→ 3 excellent
Notes:
Formulation A was considered by the testers to be generally comparible
to the skin as a commercially used baby lotion (oinment), in particular
after 15 minutes from application time.

### Example 3

### Foamable Oil in Water Emulsion Vehicle Compositions, Containing PPG Alky Ether

PART A - 15% PPG

| | 1 | 2 | 3 | 4 |
|---|---|---|---|---|
| Ingredient | % w/w | % w/w | % w/w | % w/w |
| PPG-15 stearyl ether | 15.00 | 15.00 | 15.00 | 15.00 |
| Isopropyl miristate (emollient) | — | — | 3.00 | — |
| Lanolin (emollient) | — | 2.00 | — | — |
| Behenyl alcohol (foam adjuvant) | 1.00 | 1.00 | 1.00 | 1.00 |
| Steareth-21 (surfactant) | 1.50 | — | — | — |
| Ceteareth 20 (surfactant) | — | 1.50 | 1.50 | 1.50 |
| Ceteth 2 (surfactant) | — | — | 2.00 | 2.00 |
| Laureth-4 (surfactant) | 2.00 | 2.00 | — | — |
| Carboximethyl cellulose sodium (thickener) | — | 0.50 | — | — |
| Carbomer 1342 (thickener) | — | — | 0.05 | 0.05 |
| Methyl cellulose (thickener) | 0.15 | — | 0.15 | 0.15 |
| Xanthan gum (thickener) | 0.15 | — | — | — |
| Glycerin USP (thickener) | 3.00 | 3.00 | 3.00 | 3.00 |
| Polyethylene glycol 400 (thickener) | — | 5.00 | — | — |
| Propylene glycol (polar solvent) | 5.00 | — | 5.00 | 5.00 |
| Sol. of NaOH (base) | to pH 8.5 | to pH 8.5 | to pH 8.5 | to Ph 8.5 |
| Purified water | To 100 | To 100 | To 100 | To 100 |

PART-B - 15% PPG

| | 5 | 6 | 7 |
|---|---|---|---|
| Ingredient | % w/w | % w/w | % w/w |
| PPG-15 stearyl ether | 15.00 | 15.00 | 15.00 |
| Lanolin | — | — | 2.00 |
| Steareth-21 | — | 1.50 | — |
| Behenyl alcohol | 1.00 | 1.00 | 1.00 |
| Laureth-4 | — | 2.00 | 2.00 |
| Macrogolryl Cetostearyl Ether | — | — | 1.50 |
| Ceteareth 20 | 1.60 | — | — |
| Ceteth 2 | 2.00 | — | — |
| Pemulen TR2 | 0.05 | — | — |
| Methocel A4M | 0.16 | 0.15 | — |
| CMC Sodium | — | — | 0.50 |
| Xanthan Gum | — | 0.15 | — |
| Glycerin USP | 3.00 | 3.00 | 3.00 |
| Propylene glycol | 5.40 | 5.40 | 5.00 |
| Lactic acid | To pH 7.5 | To pH 7.5 | To pH 7.5 |
| Purified Water | To 100 | To 100 | To 100 |
| Propellant | 8.00 | 8.00 | 8.00 |
| Foam Quality T-0 | E | E | E |
| Foam Quality FTC | E | E | E |
| Density T-0 | 0.043 | 0.038 | 0.040 |
| Density FTC | 0.038 | 0.034 | 0.034 |

Notes:
The liquefied or gas propellant can be added at a concentration of about 3% to about 25%.
The compositions contain a variety of organic carriers, in addition to the PPG alkyl ether.
In the majority of the compositions the surface active agents are solely non-ionic.
The formulations contain polar solvents, which contribute to skin penetration of an active agent.
Formula 1 was found to be non flammable when subjected to an aerosol inflammability test which was performed dalong the lines of European Standard prEN 14851, titled “Aerosol containers - Aerosol foam flammability test” although in a simplified form. According to this standard, a product is considered inflammable if a stable flame appears following ignition, which is at least 4 cm high and which is maintained for at least 2 seconds. Approximately 5 g of foam, mousse gel or paste is sprayed from the aerosol container on to a watchglass. An ignition source (a lighter) was placed at the base of the watchglass and any ignition and sustained combustion of the foam, mousse, gel or paste was observed. The test was carried out in a draught-free vironment capable of ventilation, with the temperature controlled at 20 ± 5° C. and relative humidity in the rangle of 30% to 80%. According to the standar, appearance of a stable flame whic

simplified form. According to this standard, a product is considered inflammable if a stable flame appears following ignition, which is at least 4 cm high and which is maintained for at least 2 seconds. Approximately 5 g of foam, mousse gel or paste is sprayed from the aerosol container on to a watchglass. An ignition source (a lighter) was placed at the base of the watchglass and any ignition and sustained combustion of the foam, mousse, gel or paste was observed. The test was carried out in a draught-free environment capable of ventilation, with the temperature controlled at 20±5° C. and relative humidity in the range of 30% to 80%. According to the standard, appearance of a stable flame which is at least 4 cm high and which is maintained for at least 2 seconds defines a product as “inflammable.”

### Example 4

### Foamable Oil in Water Emulsion Vehicle Compositions, Containing Lower Levels of PPG Alky Ether (3%)

| | 8 | 9 | 10 |
|---|---|---|---|
| Ingredient | % w/w | % w/w | % w/w |
| PPG-15 stearyl ether | 3.00 | 3.00 | 3.00 |
| Octyldodecanol (emollient) | 12.00 | 12.00 | 12.00 |
| Lanolin (emollient) | 2.00 | — | — |
| Behenyl alcohol (foam adjuvant) | 1.00 | 1.00 | — |
| Steareth-21 (surfactant) | — | — | 1.50 |
| Ceteareth-20 (surfactant) | 1.50 | 1.50 | — |
| Ceteth-2 (surfactant) | — | 2.00 | — |
| Laureth-4 (surfactant) | 2.00 | — | 2.00 |
| Carbomer 1342 (thickener) | — | 0.05 | — |
| Carboxymethyl cellulose Sodium (thickener) | 0.50 | — | — |
| Methyl cellulose (thickener) | — | 0.15 | 0.15 |
| Xanthan gum (thickener) | | — | 0.15 |
| Tromethamine buffering agent) | — | 0.50 | 0.50 |
| Disodium phosphate dehydrate | 0.25 | — | — |
| (buffering agent) | | | |
| EDTA-sodium (stabilizer) | 0.05 | 0.05 | 0.05 |
| Dichlorobenzyl alcohol (preservative) | 0.10 | 0.10 | 0.10 |
| Diazolidinyl urea (preservative) | 0.30 | 0.30 | 0.30 |
| Glycerin (polar solvent) | 3.00 | 3.00 | 3.00 |
| Propylene glycol (polar solvent) | — | 5.00 | 5.00 |
| PEG-400 (polar solvent) | 5.00 | — | — |
| Purified water | To 100 | To 100 | To 100 |
| Propellant | | | |

Notes:
The liquefied or gas propellant can be added at a concentration of about 3% to about 25%.
The compositions contain a variety of organic carriers, in addition to the PPG alkyl ether.
In the majority of the compositions the surface active agents are solely non-ionic.
The formulations contain polar solvents, which contribute to skin penetration of an active agent.

### Example 5

### Foamable Oil in Water Emulsion Vehicle Compositions, Containing PPG Alky Ether and a Vitamin D Derivative (3%, 10% and 15% Ppg)

| Ingredient | % w/w | % w/w | % w/w |
|---|---|---|---|
| Calcipotriol | 0.005 | 0.005 | 0.005 |
| PPG-15 stearyl ether | 10.00 | 15.00 | 3.00 |
| Octyldodecanol | — | — | 12.00 |
| Isopropyl myristate | 5.00 | 3.00 | 5.00 |
| Lanolin | 2.00 | — | 2.00 |
| Behenyl alcohol | 1.00 | 1.00 | 1.00 |
| Steareth-21 | — | — | — |
| Ceteareth-20 | 1.50 | 1.50 | 1.50 |
| Ceteth-2 | — | 2.00 | — |
| Ceteth-20 | — | — | — |
| Laureth-4 | 2.00 | — | 2.00 |
| Carbomer 1342 | — | 0.05 | — |
| Carboxymethyl cellulose Sodium | 0.50 | — | 0.50 |
| Methyl cellulose | — | 0.15 | — |
| Tromethamine | — | 0.50 | — |
| Disodium phosphate dihydrate | 0.25 | — | 0.25 |
| Disodium edetate | 0.05 | 0.05 | 0.05 |
| Dichlorobenzyl alcohol | 0.10 | 0.10 | 0.10 |
| Diazolidinyl urea | 0.30 | 0.30 | 0.30 |
| Glycerin | 3.00 | 3.00 | 3.00 |
| Propylene glycol | — | 5.00 | — |
| PEG-400 | 5.00 | — | 5.00 |
| Sol. of NaOH | To pH | To pH | To pH |
| | 8.5 ± 0.5 | 8.5 ± 0.5 | 8.5 ± 0.5 |
| Purified water | To 100 | To 100 | To 100 |
| Propellant | 8.00 | 8.00 | 8.00 |

Notes:
The liquefied or gas propellant can be added at a concentration of about 3% to about 25%.
The compositions contain a variety of organic carriers, in addition to the PPG alkyl ether.
In the majority of the compositions the surface active agents are solely non-ionic.
The formulations contain polar solvents, which contribute to skin penetration of an active agent.
The formulation is suitable for the treatment of psoriasis and any other condition which is responsive to treatment with a vitamin D derivative.

### Example 6

### Foamable Oil in Water Emulsion Vehicle Compositions, Containing PPG Alky Ether and Permethrin

| Ingredient | % w/w |
|---|---|
| Permethrin | 5.00 |
| PPG 15 stearyl ether | 15.00 |
| Isopropyl myristate (emollient) | 5.00 |
| Ceteareth-20 (surfactant) | 3.30 |
| PEG 40 Stearate (surfactant) | 2.00 |
| Benzyl alcohol (preservative) | 1.50 |
| Stearyl alcohol (foam adjuvant) | 1.10 |
| Carboxymethyl cellulose (thickener) | 0.55 |
| Glyceryl monostearate (co-emulsifier) | 0.50 |
| Propylene glycol (polar solvent) | 3.30 |
| Purified water | To 100 |
| Propellant | 8.00 |

| Properties of Pre Foam | | |
|---|---|---|
| Formulation | At time = 0 | After FTC |
| Centrifugation 3k | 98 | NM |
| Centrifugation 10k | 90 | |
| Viscosity | 1755 | |
| pH directed | 6.74 | |
| pH diluted 1:5 | 6.92 | |
| Foam Properties | | |
| Quality | E | E |
| color | white | white |
| Odor | Faint odor | Faint odor |
| Density | 0.041 | 0.048 |
| pH diluted 1:5 | 6.85 | 6.85 |
| Expansion time | 80 sec | NA |
| Collapse time | >300 | NA |

E = Excellent,
NM = Not measured
Notes:
The liquefied or gas propellant can be added at a concentration of about 3% to about 25%.
The compositions contain a variety of organic carriers, in addition to the PPG alkyl ether.
The permethrin concentration can range between about 1% and about 8%.
In the majority of the compositions the surface active agents are solely non-ionic.
The formulations contain polar solvents, which contribute to skin penetration of an active agent.

### Example 7

### Oil in Water Foamable Compositions Comprising PPG

PART I: 5-15% PPG

| Ingredient: | % w/w | % w/w | % w/w | % w/w | % w/w | % w/w | % w/w | % w/w |
|---|---|---|---|---|---|---|---|---|
| Active | 1.00 | 1.00 | 1.00 | 1.00 | 1.00 | 1.00 | 1.00 | 1.00 |
| Ingredient | | | | | | | | |
| PPG-15 | 15.00 | 5.00 | 15.00 | 5.00 | 15.00 | 15.00 | 15.00 | 7.00 |
| stearyl ether | | | | | | | | |
| Behenyl | 1.00 | — | 1.00 | — | 1.00 | 1.00 | — | — |
| alcohol | | | | | | | | |
| Glyceryl | — | 0.65 | — | — | — | — | — | 0.65 |
| monostearate | | | | | | | | |
| Glycerin | 3.00 | — | 3.00 | — | 3.00 | 3.00 | 3.00 | — |
| anhydrous | | | | | | | | |
| Lanolin | — | — | — | — | — | 2.00 | — | — |
| Sorbitan | — | 0.65 | — | — | — | — | — | 0.65 |
| stearate | | | | | | | | |
| Stearyl alcohol | — | 0.92 | — | 1.00 | — | — | 1.00 | 0.92 |
| Benzyl alcohol | — | — | 1.00 | — | — | — | — | — |
| Ceteareth-20 | 1.60 | — | 1.60 | — | — | 1.50 | — | — |
| Seteareth-21 | | 2.20 | | 2.00 | 1.50 | — | 1.50 | 2.20 |
| Ceteth-2 | 2.00 | — | 2.00 | 2.00 | — | — | — | — |
| Laureth-4 | — | — | — | — | 2.00 | 2.00 | 2.00 | — |
| PEG-40 | 2.85 | 2.85 | 2.85 | — | — | — | — | 2.85 |
| stearate | | | | | | | | |
| Xanthan gum | 0.30 | 0.30 | 0.30 | 0.30 | 0.15 | — | 0.15 | 0.30 |
| Methocel A4M | 0.16 | 0.35 | 0.16 | 0.35 | 0.15 | — | 0.15 | 0.35 |
| Pemulen TR2 | 0.05 | — | 0.05 | — | — | — | — | — |
| CMC | — | — | — | — | — | 0.50 | — | — |
| Poropylene | 5.40 | — | 5.40 | — | 5.40 | 5.00 | 5.40 | — |
| glycol | | | | | | | | |
| Polysorbate | 1.00 | 1.00 | 1.00 | 1.00 | — | | | 1.00 |
| 80 | | | | | | | | |
| Lactic acid | to pH | to pH | to pH | to pH | to pH | to pH | to pH | to pH |
| | 7.0 | 7.0 | 7.0 | 7.0 | 7.0 | 7.0 | 7.0 | 7.0 |
| Purified water | To | To | To | To 100 | To | To | To | To |
| | 100 | 100 | 100 | | 100 | 100 | 100 | 100 |
| Propellant | 8.00 | 8.00 | 8.00 | 8.00 | 8.00 | 8.00 | 8.00 | 8.00 |
| Foam Quality | E | G | E | G | E | E | E | E |

E = Excellent; G = Good

PART - II - 3-10% PPG

| Ingredient | % w/w | % w/w | % w/w | % w/w | % w/w | % w/w |
|---|---|---|---|---|---|---|
| Active Ingredient | 0.005 | 0.005 | 0.005 | 0.005 | 0.005 | 0.005 |
| Propylene glycol | 5.00 | — | — | — | — | 5.00 |
| PEG-400 | — | 5.00 | 5.00 | 5.00 | 5.00 | — |
| PPG-15 stearyl ether | 15 | 15 | 3 | 10 | 3 | 3 |
| Octyldodecanol | — | — | 12 | — | 12 | 12 |
| Isopropyl myristate | — | — | — | 5 | 5 | 5 |
| Lanolin | — | 2 | 2 | 2 | 2 | — |
| Behenyl alcohol | 1 | 1 | 1 | 1 | 1 | 1 |
| Steareth-21 | 1.5 | — | — | — | — | 1.5 |
| Ceteareth-20 | — | 1.5 | 1.5 | 1.5 | 1.5 | — |
| Ceteth-20 | 1.5 | 1.5 | — | — | — | — |
| Laureth-4 | 2 | 2 | 2 | 2 | 2 | 2 |
| Dichlorobenzyl alcohol | 0.1 | 0.1 | 0.1 | 0.1 | 0.1 | 0.1 |
| Carboxymethylcellulose | — | 0.5 | 0.5 | 0.5 | 0.5 | — |
| sodium | | | | | | |
| Methyl cellulose | 0.15 | — | — | — | — | 0.15 |
| Xanthan gum | 0.15 | — | — | — | — | 0.15 |
| Tromethamine | 0.5 | — | — | — | — | 0.5 |
| Disodium phosphate | — | 0.25 | 0.25 | 0.25 | 0.25 | — |
| dihydrate | | | | | | |
| Disodium edetate | 0.05 | 0.05 | 0.05 | 0.05 | 0.05 | 0.05 |
| dihydrate | | | | | | |
| Diazolidinyl urea | 0.3 | 0.3 | 0.3 | 0.3 | 0.3 | 0.3 |
| Glycerin | 3 | 3 | 3 | 3 | 3 | 3 |
| NaOH, 18% solution | to pH | to pH | to pH | to pH | To pH | To pH |
| | 8.5 ± 0.5 | 8.5 ± 0.5 | 8.5 ± 0.5 | 8.5 ± 0.5 | 8.5 ± 0.5 | 8.5 ± 0.5 |
| Propellant | 8.00 | 8.00 | 8.00 | 8.00 | 8.00 | 8.00 |
| Purified water | To 100 | To 100 | To 100 | To 100 | To 100 | To 100 |
| Foam Quality | E | E | E | E | E | E |
| Density | 0.032 | 0.0384 | 0.038 | 0.037 | 0.037 | 0.043 |

E = Excellent

PART III - 3% PPG

| | A | B | C |
|---|---|---|---|
| Ingredients | w/w % | w/w % | w/w % |
| Petrolatum | 2.50 | — | — |
| Mineral oil Heavy | | 5.00 | 10.00 |
| Cetearyl octanoate | 1.50 | — | — |
| Octyl dodecanol | 6.00 | — | — |
| PPG 15 Stearyl Ether | 3.00 | 3.00 | 3.00 |
| Ceteareth 20 | 3.00 | 3.00 | — |
| Cetostearyl alcohol | 1.00 | 1.00 | — |
| Stearic Acid | — | — | 1.00 |
| Glyceryl monostearate | 1.00 | — | — |
| PEG 40 stearate | — | — | 2.50 |
| Dimethicone 350 | — | 1.00 | 1.00 |
| Cyclomethicone | 1.00 | | — |
| Benzyl alcohol | 0.60 | 0.60 | 0.60 |
| Dimethicone copolyol | — | — | 1.00 |
| Polysorbate 80 | — | — | 1.00 |
| Carboxymethyl cellulose | 0.50 | 0.50 | — |
| sodium | | | |
| Hypermellose K100M | — | — | 0.25 |
| Xanthan gum | — | — | 0.25 |
| Purified water | 63.90 | 69.90 | 63.40 |
| Glycerin | 5.00 | 5.00 | 5.00 |
| Ethanol | 10.00 | 10.00 | 10.00 |
| Mentha Piperita oil | 1.00 | 1.00 | 1.00 |
| Total | 100.00 | 100.00 | 100.00 |
| Propellant | 8.00 | 8.00 | 8.00 |

| | | Appearance | | | | | | | | | |
|---|---|---|---|---|---|---|---|---|---|---|---|
| | | Quality [scoring] | | Color [scoring] | | Odor [scoring] | | Density {g/mL) | | pH 1:5 diluted with water | |
| Formula Name | Manufacturing date | T-0 | FTC | T-0 | FTC | T-0 | FTC | T-0 | FTC | T-0 | FTC |
| A | 06.12.24 | E | E | 1 | 1 | 0 | 0 | 0.044 | 0.043 | 6.55 | 6.52 |
| B | 06.12.26 | E | E | 1 | 1 | 0 | 0 | 0.039 | 0.036 | 6.55 | 6.42 |
| C | 06.12.28 | E | E | 1 | 1 | 0 | 0 | 0.036 | 0.042 | 6.06 | 5.99 |

Colour code 1 = white to yellowish

PART IV - 15%

| | PPG002 |
|---|---|
| PPG 15 stearyl ether | 15.00 |
| Glyceryl Monostearate | 0.49 |
| Sorbitane Stearate | 0.65 |
| Stearyl Alcohol | 0.92 |
| Steareth-21 | 2.17 |
| PEG-40 Stearate | 2.83 |
| Methocel A4M | 0.33 |
| Xanthan gum | 0.28 |
| Polysorbate 80 | 0.98 |
| Water purified | 76.35 |
| Total | 100.00 |
| Propellant (1681) | 8.00 |
| Foam quality | E |
| Color | White |
| Odor | No |
| Shakability | Good |

E = Excellent
Notes:
The liquefied or gas propellant can be added at a concentration of about 3% to
about 25%.
In the majority of the compositions the surface active agents are solely non-ionic.

### Example 8

### Waterless PPG Foamable Compositions Having Small Amounts of PPG (≦15%)

PART A: 15% PPG

| Ingredient | % w/w |
|---|---|
| Active | 0.069 |
| ingredient | |
| Glycerin | 33.00 |
| Hydroxypropyl | 3.00 |
| cellulose | |
| Stearyl alcohol | 2.00 |
| Steareth-2 | 2.00 |
| PEG 100 | 3.00 |
| stearate & | |
| glyceryl stearate | |
| PPG-15 Stearyl | 15.00 |
| ether | |
| Ceteareth-20 | 2.00 |
| Propylene glycol | To |
| | 100 |
| Foam Quality | G |
| Density | 0.195 |

PART B: 3% and 15% PPG

| Ingredient name (INCI, CTFA) | A | B |
|---|---|---|
| Propylene glycol | % w/w | % w/w |
| Glycerin USP | 43 | 88.35 |
| Stearyl Alcohol | 33 | |
| Hydroxypropyl Cellulose | 1 | 1.94 |
| Laureth-4 | 3 | 1.94 |
| GMS NE | | 1.94 |
| Steareth 2 | | 1.94 |
| Simulsol 165 | 2 | |
| PPG-15 stearyl ether | 15 | 2.91 |
| Macrogol Cetostearyl Ether | 2 | 0.97 |
| Control: | 100 | 100 |
| Propellant | 8 | 8 |
| Shaking | 5 | 6 |
| Separation | Y | Y |

G = Good
Notes:
The liquefied or gas propellant can be added at a concentration of about 3% to about 25%.

### Example 9

### Theoretical Aqueous PPG Formulations with Active Agent

Exemplary concentrations of active agents in foamable compositions are set out in Table 2. Each active agent is added into, for example, any of the carriers listed in any of the above aqueous Examples in a therapeutically effective concentration and amount. The methodology of addition is well known to those of the art. The composition is adjusted in each case so that it is made up to 100% w/w as appropriate by water.

TABLE 2
Exemplary Concentrations of Examples of Active Agents

| Class | Concentration | Exemplary Use |
|---|---|---|
| Hydrocortisone | 1% | Steroid responsive inflammation and |
| acetate | | |
| Betamethasone | 0.12% | psoriasis or atopic dermatitis |
| valerate | | |
| Clobetasol | 0.05% | |
| proprionate | | |
| Acyclovir | 5% | Viral infection, herpes |
| Ciclopirox | 1% | Fungal infection, seborrhea, dandruff, |
| Clindamycin | 1-2% | Bacterial infection, acne, rosacea, |
| Azelaic acid | 15% | Acne, rosacea, |
| | | pigmentation disorder and various |
| | | dermatoses |
| Metronidazol | 0.25%-2% | Rosacea, bacterial infections and |
| | | parasite infestations |
| Diclofenac | 1% | Osteoarthritus, joint pain |
| Tacrolimus | 0.2% | Atopic dermatitis, eczema and |
| | | inflammation |
| Caffeine | 5% | anti-cellulite |
| Clotrimazole | 1% | Fungal infection |
| Lidocaine base | 2% | Local anaesthetic |
| Terbinafine HCL | 1% | Fungal infection |
| Gentamycin | 0.1% | Bacterial skin infections, burns or |
| | | ulcers |
| Dexpanthenol | 5% | Wounds, ulcers, minor skin infections |
| Urea | 5-10% | Emollient and keratolytic |
| | | Atopic dermatitis, eczema, ichthyosis |
| | | and hyperkeratotic skin disorders |
| Ammonium | 12%-17.5% | Dry scaly conditions of the skin |
| lactate | | including ichthyosis |
| Povidone-iodine | 10% | Antimicrobial-antiseptic |

Note, all the above active agents have a degree of solubility in water or petrolatum or the composition other than clobestol proprionate, which is practically insoluble; tacrolimus, which is insoluble in water; and betamethasone valerate which although has very limited solubility is nevertheless, surprisingly soluble at least to a degree in the compositions of the present invention, in the water phase.

The above examples represent different drug classes and it is to be understood that other drugs belonging to each of the classes represented above or described elsewhere in the specification may be included and used in the compositions of the present invention in a safe and effective amount.

### Example 10

### Theoretical Non Aqueous PPG Formulations with Active Agent

Exemplary concentrations of active agents in foamable compositions are set out in Table 2 above. Each active agent is added into, for example, any of the carriers listed in any of the above Non Aqueous Examples in a therapeutically effective concentration and amount. The methodology of addition is well known to those of the art. The composition is adjusted in each case so that it is made up to 100% w/w as appropriate by solvent or petrolatum.

### Example 11

### Theoretical of Substantially Non Aqueous PPG Formulations with Active Agent

Exemplary concentrations of active agents in foamable compositions are set out in Table 2 above. Each active agent is added into, for example, any of the carriers listed in any of the above substantially Non Aqueous Examples in a therapeutically effective concentration and amount. The methodology of addition is well known to those of the art. The composition is adjusted in each case so that it is made up to 100% w/w as appropriate by water, solvent or petrolatum.

1. A foamable pharmaceutical carrier comprising:
a polypropylene glycol alkyl ether of about 3% to about 90% by weight of the total composition;
a surface-active agent;
a solvent; and
a liquefied or compressed gas propellant at a concentration of about 3% to about 25% by weight of the total composition.

2-300. (canceled)
